(19) United States
(12) Patent Application Publication
(10) Pub. No.: US 2024/0309461 A1
(43) Pub. Date: Sep. 19, 2024

(54) SAMPLE BARCODE IN MULTIPLEX SAMPLE SEQUENCING

(71) Applicant: GRAIL, LLC, Menlo Park, CA (US)

(72) Inventors: Seyedmedhi Shojaee, San Francisco, CA (US); Nathan Hunkapiller, Belmont, CA (US); Byoungsok Jung, Atherton, CA (US); Farnaz Absalan, San Carlos, CA (US); Chenlu Hou, San Carlos, CA (US); Sahar Nohzadeh-Malakshah, Union City, CA (US); Christopher Chang, Palo Alto, CA (US)

(21) Appl. No.: 18/603,522

(22) Filed: Mar. 13, 2024

Related U.S. Application Data
(60) Provisional application No. 63/489,848, filed on Mar. 13, 2023.

(51) Int. Cl. C12Q 1/6886; C12N 15/10; C12Q 1/6874; G16B 30/00; G16B 40/20

(52) U.S. Cl. CPC C12Q 1/6886; C12N 15/1065; C12Q 1/6874; G16B 30/00; G16B 40/20; C12Q 2600/154

(57) ABSTRACT

Methods and systems for sample contamination detection are disclosed. In particular, sample barcodes are utilized, wherein each sample barcode is assigned to a sample and ligated to fragments from the sample. The sample barcodes are used in conjunction with indices from sequencing libraries to accurately assign sequence reads to samples during multiplex sequencing. Molecule identifiers may also be utilized to aid in de-duping of sequence reads to precisely identify original NA fragments from a sample. Accordingly, in one or more embodiments, a sequencing method includes isolating DNA fragments in a sample, ligating the DNA fragments with unique molecule identifiers (UMIs), performing an amplification process resulting in amplicons, ligating a sample barcode onto the amplicons, and performing amplicon sequencing. The analytics system looks to whether indices are matched and whether a sample barcode matches to the pair of indices when identifying single-index or double-index hopping events.

### CROSS-REFERENCE TO RELATED APPLICATIONS

The present application claims the benefit of and priority to U.S. Provisional Application No. 63/489,848 filed on Mar. 13, 2023, which is incorporated by reference.

### BACKGROUND

Cancer is a leading cause of death worldwide. The fatality of cancer is heightened by the fact that cancer is usually detected in latter stages, limiting efficacy of treatment options for long-term survival. Current detection methods generally are cancer type specific, i.e., each cancer type is individually screened for. Each individual screening process is tailored to the cancer type. For example, mammography scans are utilized in breast cancer detection, whereas colonoscopy or fecal tests have helped with colorectal cancer detection. Each varied screening method is not cross-applicable to other cancer types. For example, to screen one individual for three different possible cancer types, a healthcare provider would need to perform or order to be performed three different screening processes. Each of those screening processes may entail a combination of invasive and/or non-invasive procedures to identify tumorous growths, collect a biopsy of the growth, and perform analysis on the tissue biopsy.

Furthermore, present screening methods are encumbered by low detection rates or high false positive rates. Low detection rates often fail to detect early-stage cancers as the cancers are just developing. A high positive rate misdiagnoses cancer-free individuals as positive for cancer status. As a result, most screening tests are only practical when they are used to test individuals who have a high risk of developing the screened cancer, and they have limited ability to detect cancers in the general population.

Novel research has implicated aberrant DNA methylation in many disease processes, including cancer. DNA methylation plays a role in regulating gene expression. Thus, aberrant DNA methylation can create issues in normal gene expression pathways, thereby leading to cancer or other diseases. For example, specific patterns of differentially methylated regions may be useful as molecular markers for various disease states. Nonetheless, even such models face a number of challenges. Early cancer detection is particularly challenging due to the miniscule ratio of tumor cells to non-cancer cells in the subject. The miniscule ratio may be on the order of 1:1000, 1:10,000, or even 1:100,000. This creates a challenge of detecting small amounts of cancer signal amidst healthy signal. Moreover, DNA may be shed by blood cells which may comprise age-related genetic variations, often resembling cancerous aberrant methylation. These informatively methylated fragments shed from blood cells can often ostensibly inflate cancer signal.

In one or more implementations, the early cancer detection workflow implements next-generation sequencers capable of high-throughput sequencing. In such implementations, these sequencing devices may include multiplex sequencing capabilities for sequencing molecules from multiple samples. To aid in multiplex sequencing, a library of indices is used to tag and distinguish the sequence reads between the various samples. However, index hopping events in the sequencing process can cause misassignment of sequence reads to the wrong sample. Such misassignment of reads can skew downstream analyses, e.g., inaccurately detecting cancer detection in one sample based on misassigned sequence reads from another sample.

The present disclosure is directed to addressing the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of a somatic variant, methylation status, or other genetic aberrations) from a blood sample can provide insight into whether a subject may have cancer, and further insight on what type of cancer the subject may have. Towards that end, this description includes systems and methods for contamination detection of nucleic acid samples for the purpose of analyzing the sequencing data for cancer assessment.

The present disclosure addresses the problems identified above by providing improved systems and methods for sample contamination detection of contaminated fragments for cancer classification. The system utilizes substantially unique sample barcode sequences that are ligated to nucleic acid (NA) molecules originating from one sample. The sample barcode sequences in addition to the one or more indices in a sequencing library help to accurately assign sequence reads to the appropriate sample, thereby providing an improvement to next-generation sequencing technology and, more generally, to the physical assaying process.

The analytics system initially evaluates the indices of the sequence reads from a multiplex sequencing process. Sequence reads with similar indices are collated together. Sequence reads with mismatched indices (two indices not paired in any sequencing library) can be determined to be single-index hopping events. Sequence reads with matched indices but a different sample barcode are determined to be double-index hopping events. In instances where indices and/or sample barcode sequences are different but close, the analytics system may determine identify sequencing errors.

In one or more embodiments, NA molecules in a sample are also ligated with unique molecule identifiers (UMIs), which aid in accurate de-duping of sequence reads belonging to the same original NA molecule. Various configurations of sample barcodes, UMIs, and indices can be implemented. In one or more configuration, the sample barcode and the UMI on each NA molecule are on opposite ends of the NA molecule. In other configurations, the sample barcode and the UMI are ligated on the same end of the NA molecule. Additional configurations alternatively utilize single-index sequencing libraries or double-index sequencing libraries.

Clause 1. A method comprising: ligating one of a plurality of molecule identifiers (MIs) to a first end of each nucleic acid (NA) fragment of a first sample, wherein at least two of the plurality of MIs are different from one another; amplifying the NA fragments to produce amplified NA fragments comprising one or more copies of each NA fragment; ligating a first sample barcode to the amplified NA fragments of the first sample; indexing the amplified NA fragments to produce indexed NA fragments each comprising a first index and a second index; sequencing the indexed NA fragments to generate a sequence read for each indexed NA fragment; collecting sequence reads of indexed NA fragments each comprising a first index sequence read and a second index sequence read in a group; and identifying a contamination event for a first sequence read in the group by identifying a second sample barcode that is different than the first sample barcode on the sequence read.

Clause 2. The method of clause 1 or any clause dependent thereon, wherein each amplified NA fragment having the first sample barcode comprises a target NA region derived from the sample and wherein each amplified NA fragment having the second sample barcode comprises a target NA region derived from a second sample that is different than the first sample.

Clause 3. The method of clause 1 or any clause dependent thereon, further comprising removing the first sequence read with the index hopping event from the group for the first sample.

Clause 4. The method of clause 1 or any clause dependent thereon, further comprising generating a binary cancer prediction between presence of cancer and absence of cancer based on the sequence reads in the group excluding the first sequence read.

Clause 5. The method of clause 1 or any clause dependent thereon, further comprising generating a multiclass cancer prediction between a plurality of cancer types based on the sequence reads in the group excluding the first sequence read.

Clause 6. The method of clause 1 or any clause dependent thereon, wherein the one of the plurality of MIs and the NA fragment are single-stranded during ligation.

Clause 7. The method of clause 1 or any clause dependent thereon, wherein the indexed NA fragments each comprise the first index at a first end and the second index at a second end.

Clause 8. The method of clause 1 or any clause dependent thereon, wherein the first end is a 3′ end.

Clause 9. The method of clause 1 or any clause dependent thereon, wherein the first end is a 5′ end.

Clause 10. The method of clause 1 or any clause dependent thereon, wherein the first sample barcode is ligated to a second end of the amplified NA fragments opposite the first end.

Clause 11. The method of clause 1 or any clause dependent thereon, wherein the first sample barcode is ligated to the first end of the amplified NA fragments, adjacent to the one of the plurality of MIs.

Clause 12. The method of clause 1 or any clause dependent thereon, wherein the first index is ligated to the first end, and the second index is ligated to a second end that is opposite the first end.

Clause 13. The method of clause 1 or any clause dependent thereon, wherein the first index and the second index are ligated to the first end of the amplified NA fragments.

Clause 14. The method of clause 1 or any clause dependent thereon, wherein the first index and the second index are ligated to a second end of the amplified NA fragments that is opposite the first end.

Clause 15. The method of clause 1 or any clause dependent thereon, wherein the sequencing is multiplexed with a plurality of samples across a plurality of flow cells, and wherein the first index and the second index are used for a first column that the first sample is in.

Clause 16. The method of clause 1 or any clause dependent thereon, wherein a third index and a fourth index, that are different from the first index and the second index, are used for a second column.

Clause 17. The method of clause 1 or any clause dependent thereon, wherein the sequencing comprises a targeted methylation sequencing or a whole genome bisulfite sequencing.

Clause 18. The method of clause 1 or any clause dependent thereon, wherein each MI has a length selected from a range of 3 nucleobases to 20 nucleobases.

Clause 19. The method of clause 1 or any clause dependent thereon, wherein the first sample barcode has a length selected from a range of 3 nucleobases to 20 nucleobases.

Clause 20. The method of clause 1 or any clause dependent thereon, wherein the first sample barcode is substantially unique from a plurality of sample barcodes.

Clause 21. The method of clause 1 or any clause dependent thereon, wherein the NA fragments are cell-free deoxyribonucleic acid (cfDNA) fragments.

Clause 22. The method of clause 1 or any clause dependent thereon, wherein amplifying the NA fragments comprises performing linear amplification.

Clause 23. A method for calling a contamination event, the method comprising: receiving a plurality of sequence reads of amplified fragments, wherein each of the amplified fragments comprises a first index, a second index, and a sample barcode; collating sequence reads of amplified fragments having a first index and a second index in a first bag for a first sample with a first sample barcode assigned to the first sample; and calling the contamination event for a first sequence read in the first bag based on identifying a second sample barcode that is different than the first sample barcode on the sequence read.

Clause 24. The method of clause 23 or any clause dependent thereon, wherein the first index is on a first end of an amplified fragment and the second index is on a second end of the amplified fragment.

Clause 25. The method of clause 23 or any clause dependent thereon, wherein the first index and the second index are on a first end of an amplified fragment.

Clause 26. The method of clause 23 or any clause dependent thereon, wherein each of the amplified fragments further comprises a molecule identifier (MI), wherein amplified fragments derived from a first nucleic acid fragment of a sample comprises a first MI, and amplified fragments derived from a second nucleic acid fragment of the sample comprises a second MI that is different that the first MI.

Clause 27. The method of clause 23 or any clause dependent thereon, wherein the MIs are on a first end of the amplified fragments with the sample barcode ligated on the first end.

Clause 28. The method of clause 23 or any clause dependent thereon, wherein the MIs are on a first end of the amplified fragments with the sample barcode ligated on a second end that is opposite the first end.

Clause 29. The method of clause 23 or any clause dependent thereon, further comprising: removing the first sequence read with the contamination event from the first bag for the first sample.

Clause 30. The method of clause 29, further comprising: generating a cancer prediction for the first sample based on the sequence reads in the first bag excluding the first sequence read.

Clause 31. The method of clause 30, wherein the cancer prediction is a binary prediction between presence of cancer and absence of cancer.

Clause 32. The method of clause 30 or any clause dependent thereon, wherein the cancer prediction is a multiclass prediction between a plurality of cancer types.

Clause 33. The method of clause 30 or any clause dependent thereon, wherein generating the cancer prediction comprises: collapsing the sequence reads into a set of distinct sequence reads; determining an informative score for each distinct sequence read, wherein the informative score indicates a likelihood of observing the distinct sequence read in a healthy population of samples; and identifying a set of informative fragments by comparing the informative scores for the distinct sequence reads against an informative score threshold, wherein the cancer prediction is based on the set of informative fragments.

Clause 34. The method of clause 33, wherein generating the cancer prediction further comprises: generating a feature vector for the first sample based on the set of informative fragments; and inputting the feature vector into a cancer classifier to determine the cancer prediction, wherein the cancer classifier is trained on at least a first cohort of cancer samples and a second cohort of non-cancer samples.

Clause 35. The method of clause 23 or any clause dependent thereon, wherein the first sample barcode has a length selected from a range of 3 nucleobases to 20 nucleobases.

Clause 36. A method for processing sequencing data, comprising: receiving sequencing data comprising a set of sequence reads generated from multiplex sequencing a plurality of biological samples, each containing nucleic acid, the sequencing data including data generated from single-index hopping and double-index hopping events arising during the multiplex sequencing; filtering the sequencing data to exclude data corresponding to the single-index hopping events, the filtering comprising: identifying, in the set of sequence reads, one or more reads having a mismatched pair of indices, the mismatched pair of indices comprising two unique indices corresponding to two different biological samples; and filtering the sequencing data to exclude data corresponding to the double-index hopping events, the filtering comprising: identifying, in the set of sequence reads, one or more pad-hopping duplicate reads, the pad-hopping duplicate reads having duplicate sequences that are co-localized in a flow cell used during the multiplex sequencing; and subsequent to identifying the one or more pad-hopping duplicate reads, identifying, in the set of sequence reads, one or more singletons, each singleton comprising a unique sequence read among the set of sequence reads.

Clause 37. The method of clause 36 or any clause dependent thereon, comprising: removing the identified one or more pad-hopping duplicate reads from the set of sequencing reads; and subsequent to removing the identified one or more pad-hopping duplicate reads, identifying the one or more singletons in the remaining set of sequencing reads.

Clause 38. The method of clause 36 or any clause dependent thereon, wherein filtering comprises: flagging, in the sequencing data, the identified one or more reads having mismatched indices, the identified one or more pad-hopping duplicate reads, or the identified one or more singletons.

Clause 39. The method of clause 36 or any clause dependent thereon, wherein filtering comprises: removing, from the sequencing data, the identified one or more reads having mismatched indices, the identified one or more pad-hopping duplicate reads, or the identified one or more singletons.

Clause 40. The method of clause 36 or any clause dependent thereon, wherein the flow cell comprises a plurality of physically separated lanes, wherein each lane comprises multiple columns with each column comprising a plurality of tiles, further wherein each lane defines a surface having a plurality of wells arranged thereon.

Clause 41. The method of clause 36 or any clause dependent thereon, wherein the sequence data comprises spatial information indicating a location of each sequence read on the flow cell, the spatial information comprising at least one of a lane ID, a column ID, a tile ID, or an x-y coordinate pair.

Clause 42. The method of clause 36 or any clause dependent thereon, wherein identifying pad-hopping duplicate reads comprises: identifying a group of identical or nearly-identical sequence reads; determining, based on the sequence data, whether the grouped reads are co-localized, wherein the grouped reads are co-localized when at least one of the following positional relationships is met: the grouped reads share a common tile, the grouped reads are located in neighboring tiles, the grouped reads are located in different tiles within a common column, the grouped reads are located within a threshold x-distance and a threshold y-distance from each other on the flow cell, and the grouped reads are located within a predefined boundary region; and in accordance with a determination that the grouped reads are co-localized, identifying the grouped reads as pad-hopping duplicate reads.

Clause 43. The method of clause 42 or any clause dependent thereon, comprising identifying multiple groups of sequence reads as pad-hopping duplicate reads, each group comprising identical or nearly-identical sequence reads that are co-localized.

Clause 44. The method of clause 42 or any clause dependent thereon, wherein the predefined boundary region comprises a geometric shape having an x distance of 7,500 flow cell position units and a y distance of 100,000 flow cell position units.

Clause 45. The method of clause 42 or any clause dependent thereon, wherein the geometric shape comprises a rectangle, wherein a longer side of the rectangle extends longitudinally along a lane in a y-direction of the flow cell.

Clause 46. The method of clause 42 or any clause dependent thereon, wherein the threshold x-distance between the grouped reads is within a range of 0-50 mm, and the threshold y-distance between the grouped reads is within a range of 0-50 mm.

Clause 47. The method of clause 36 or any clause dependent thereon, comprising: removing the pad-hopping duplicate reads when an expected error rate associated with the multiplex sequencing exceeds a threshold error rate.

Clause 48. The method of clause 36 or any clause dependent thereon, comprising: providing the filtered sequencing data for analysis using a statistical model, wherein a limit of detection associated with the filtered sequencing data is lower than a limit of detection associated with unfiltered sequencing data.

Clause 49. The method of clause 36 or any clause dependent thereon, wherein the nucleic acid comprises cell-free DNA (cfDNA) or cell-free RNA (cfRNA).

Clause 50. The method of clause 36 or any clause dependent thereon, wherein the nucleic acid comprises genomic DNA (gDNA).

Clause 51. The method of clause 36 or any clause dependent thereon, comprising: fragmenting the nucleic acid extracted from the plurality of biological samples into genomic fragments; ligating unique dual index pairs to end portions of the genomic fragments to generate multiple library fragments, wherein each unique dual index pair identifies an individual biological sample in the plurality of biological samples; enriching the library fragments by capturing certain library fragments with targeted probes and amplifying the captured library fragments within multiple wells on the flow cell, wherein each well is configured to hold a clonal cluster of amplified fragments originating from a single library fragment; sequencing the enriched fragments to produce the sequencing data comprising the set of sequence reads, each sequence read comprising a plurality of nucleotide base calls; and demultiplexing the set of sequence reads based on the unique dual index pairs to determine the original biological sample for each sequence read.

Clause 52. The method of clause 51, further comprising demultiplexing the sequencing data after filtering the sequencing data to exclude the data corresponding to the single-index hopping events and double-index hopping events.

Clause 53. A method for processing sequencing data, comprising: receiving sequencing data comprising a set of sequence reads generated from multiplex sequencing a plurality of biological samples containing nucleic acid, the sequencing data including data generated from single-index hopping and double-index hopping events arising during the multiplex sequencing; filtering the sequencing data to exclude data corresponding to the single index-hopping events, the filtering comprising: identifying, in the set of sequence reads, one or more reads having a mismatched pair of indices, the mismatched pair of indices comprising two unique indices corresponding to two different biological samples; and filtering the sequencing data to exclude data corresponding to the double-index hopping events, the filtering comprising: identifying, in the set of sequence reads, one or more singletons, each singleton comprising a unique sequence read among the set of sequence reads.

Clause 54. A method for training a cancer classifier comprising: performing next-generation multiplex sequencing of a set of training samples each with a known cancer state to obtain a plurality of sequence reads of amplified fragments, wherein each sequence read comprises a pair of indices and a sample barcode ligated onto a target region of a nucleic acid fragment; collating the sequence reads into a plurality of bags based on the pairs of indices of the sequence reads, wherein each bag includes sequence reads having a common pair of indices; detecting a cross-sample contamination event for a first sequence read having a first sample barcode different from other sequence reads in a first bag; removing the first sequence read from the first bag; assigning remaining sequence reads in each bag to one training sample; determining a feature vector for each training sample based on the sequence reads in the corresponding bag; and training the cancer classifier with the feature vectors for the training samples, wherein the trained cancer classifier is configured to predict likelihood of presence of cancer based on an input feature vector derived based on sequence reads in a test sample.

Clause 55. The method of clause 54 or any clause dependent thereon, wherein each sequence read further comprises a molecular identifier (MI) ligated onto the corresponding nucleic acid fragment, and wherein the method further comprises: collapsing sequences reads in each bag into distinct sequence reads based on the molecular identifiers, wherein sequence reads overlapping a similar genomic position and having matching molecular identifiers are determined to read on the same nucleic acid fragment.

Clause 56. The method of clause 54 or any clause dependent thereon, further comprising: assigning the first sequence read to a second bag with sequence reads in the second bag having the first sample barcode.

Clause 57. The method of clause 54 or any clause dependent thereon, wherein the cancer classifier is a machine-learning model.

Clause 58. The method of clause 54 or any clause dependent thereon, wherein the cancer classifier is trained to predict a binary prediction between presence of cancer or absence of cancer.

Clause 59. The method of clause 54 or any clause dependent thereon, wherein each training sample is known to have one of a plurality of cancer types.

Clause 60. The method of clause 59, wherein the cancer classifier is trained to predict a multiclass prediction as a likelihood of presence of one of the plurality of cancer types.

Clause 61. The method of clause 54 or any clause dependent thereon, wherein the sequence reads for each sample are methylation sequence reads, and wherein the feature vector is based on methylation sequence reads with an informative methylation pattern.

Clause 62. A non-transitory computer-readable storage medium storing one or more programs configured to be executed by one or more processors of a computer system, the one or more programs including instructions for performing the method of any of clauses 1-61.

Clause 63. A computer system comprising: one or more processors; and the non-transitory computer-readable storage medium of clause 62.

Clause 64. A treatment kit comprising: a collection vessel for collecting a biological sample from a subject; optionally, one or more reagents for isolating DNA fragments in the biological sample; optionally, a library of paired indices; a plurality of copies of a first sample barcode for ligating onto DNA fragments of the biological sample; and optionally, the non-transitory computer-readable storage medium of clause 62.

Clause 65. A set of nucleic acid (NA) constructs comprising: a first NA construct comprising: a first target NA region, a first sample-specific barcode comprising a first barcode sequence, and a first index; and a second NA construct comprising: a second target NA region, a second sample-specific barcode comprising a second barcode sequence, and the first index; wherein the first NA construct is identified as derived from a first sample based on the first sample-specific barcode, and wherein the second NA construct is identified as derived from the second sample based on a second sample-specific barcode.

Clause 66. The set of NA constructs of clause 65 or any clause dependent thereon, wherein the first sample-specific barcode and the first index are at a first end of the first NA construct

Clause 67. The set of NA constructs of clause 65 or any clause dependent thereon, wherein the first sample-specific barcode is at a first end of the first NA construct, and wherein the first index is at a second end of the first NA construct that is opposite the first end.

Clause 68. The set of NA constructs of clause 65 or any clause dependent thereon, wherein the first NA construct and the second NA construct are bagged together based on identifying the first index.

Clause 69. The set of NA constructs of clause 65 or any clause dependent thereon, wherein the first NA construct comprises a second index, and wherein the second NA construct comprises the second index.

Clause 70. The set of NA constructs of clause 65 or any clause dependent thereon, wherein the first index and the second index are at a first end of the first NA construct.

Clause 71. The set of NA constructs of clause 65 or any clause dependent thereon, wherein the first index is at a first end of the first NA construct, and wherein the second index is at a second end of the first NA construct that is opposite the first end.

Clause 72. The set of NA constructs of clause 65 or any clause dependent thereon, wherein the first NA construct and the second NA construct are bagged together based on identifying the first index and the second index.

Clause 73. The set of NA constructs of clause 65 or any clause dependent thereon, wherein the first barcode is at a first end of the first NA construct, and wherein the first NA construct further comprises a molecule identifier (MI) at a second end of the first NA construct that is opposite the first end.

Clause 74. The set of NA constructs of clause 65 or any clause dependent thereon, wherein at least one of the first NA construct and the second NA construct is an amplified construct.

Clause 75. The set of NA constructs of clause 65 or any clause dependent thereon, wherein the first sample-specific barcode is substantially unique from the second sample-specific barcode.

Clause 76. The set of NA constructs of clause 65 or any clause dependent thereon, wherein the first NA construct is constructed in a first column, and wherein the second NA construct is constructed in a second column, wherein the first index is assigned to the first column but index hopped to the second column.

### BRIEF DESCRIPTION OF DRAWINGS

FIG. 1 is an exemplary flowchart describing an overall workflow of cancer classification of a sample, according to one or more embodiments.

FIG. 2A is an exemplary flowchart describing a process of sequencing a fragment of cell-free (cf) DNA to obtain a methylation state vector, according to one or more embodiments.

FIG. 2B is an exemplary illustration of the process of FIG. 3A of sequencing a fragment of cell-free (cf) DNA to obtain a methylation state vector, according to one or more embodiments.

FIG. 3A is an exemplary flowchart describing a process of sample treatment with a sample barcode, according to one or more embodiments.

FIG. 3B is an exemplary flowchart describing a process of detecting contamination events utilizing in part the sample barcode, according to one or more embodiments.

FIG. 4A is an exemplary illustration of sample treatment, according to one or more embodiments.

FIG. 4B is an exemplary illustration of sample sequencing following sample treatment in FIG. 4A, according to one or more embodiments.

FIG. 5A is an exemplary illustration of contamination detection, according to one or more embodiments.

FIG. 5B is an exemplary illustration of nucleic acid constructs incorporating sample barcodes in a patterned flow cell, according to one or more embodiments.

FIG. 6 is an exemplary illustration of various configurations of sample barcodes and unique molecule identifiers, according to one or more embodiments.

FIG. 7 illustrates an exemplary flowchart illustrating a process 700 of identifying index hopping events, according to one or more embodiments.

FIG. 8A is an exemplary flowchart describing a process of generating a control group data structure for determining informatively methylated fragments, according to one or more embodiments.

FIG. 8B is an exemplary flowchart describing a process of determining a fragment to be informatively methylated based on the control group data structure, according to one or more embodiments.

FIG. 9A is an exemplary flowchart describing a process of training a cancer classifier, according to one or more embodiments.

FIG. 9B illustrates an example generation of feature vectors used for training the cancer classifier, according to one or more embodiments.

FIG. 10A illustrates an exemplary flowchart of devices for sequencing nucleic acid samples according to one or more embodiments.

FIG. 10B is an exemplary block diagram of an analytics system, according to one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

Early detection and classification of cancer is an important technology. Being able to detect cancer before it becomes symptomatic is beneficial to all parties involved, including patients, doctors, and loved ones. For patients, early cancer detection allows them a greater chance of a beneficial outcome; for doctors, early cancer detection allows more pathways of treatment that may lead to a beneficial outcome; for loved ones, early cancer detection increases the likelihood of not losing their friends and family to the disease.

Recently, early cancer detection technology has progressed towards analyzing genetic fragments (e.g., DNA) in a person's, for example, blood to determine if any of those genetic fragments originate from cancer cells. These new techniques allow doctors to identify a cancer presence in a patient that may not be detectable otherwise, e.g., in conventional screening processes. For instance, consider the example of a person at high risk for breast cancer. Traditionally, this person will regularly visit their doctor for a mammogram, which creates an image of their breast tissue (e.g., taking x-ray images) that a doctor uses to identify cancerous tissue. Unfortunately, with even the highest resolution mammograms, doctors are only able to identify tumors once they are approximately a millimeter in size. This means that the cancer has been present for some time in the person and has gone undiagnosed and untreated. Visual determinations like this are typical for most cancers—that is, only being identifiable once it has grown to a sufficient size and has become identifiable with some sort of imaging technology.

Cancer detection using analysis of genetic fragments in a patient's, e.g., blood alleviates this issue. To illustrate, cancer cells will start sloughing DNA fragments into a person's bloodstream as soon as they form. This occurs when there are very few of the cancer cells, and before they would be visible with imaging techniques. With the appropriate methods, therefore, a system that analyzes DNA fragments in the bloodstream could identify cancer presence in a person based on sloughed cancer DNA fragments, and, more importantly, they system could do so before the cancer is identifiable using more traditional cancer detection techniques.

Cancer detection based on the analysis of DNA fragments is enabled by next-generation sequencing (“NGS”) techniques. NGS, broadly, is a group of technologies that allows for high throughput sequencing of genetic material. As discussed in greater detail herein, NGS largely consists of (1) sample preparation, (2) DNA sequencing, and (3) data analysis. Sample preparation is the laboratory methods necessary to prepare DNA fragments for sequencing, sequencing is the process of reading the ordered nucleotides in the samples, and data analysis is processing and analyzing the genetic information in the sequencing data to identify cancer presence.

While these steps of NGS may help enable early cancer detection, they also introduce their own complex, detrimental problems to cancer detection and, therefore, any improvements to sample preparation, DNA sequencing, and/or data analysis, including the pre-processing, algorithmic processing, and summary or presentation of predications or conclusions, results in an improvement to cancer detection technologies and early cancer detection more generally.

To illustrate, as an example, problems introduced in (1) sample preparation include DNA sample quality, sample contamination, fragmentation bias, and accurate indexing. Remedying these problems would yield better genetic data for cancer detection. Similarly, problems introduced in (2) sequencing include, for example, errors in accurate transcribing of fragments (e.g., reading an “A” instead of a “C”, etc.), incorrect or difficult fragment assembly and overlap, disparate coverage uniformity, sequencing depth vs. cost vs. specificity, and insufficient sequencing length. Again, remedying any of these problems would yield improved genetic data for cancer detection.

The problems in (3) data analysis are the most daunting and complex. The introduced challenges stem from the vast amounts of data created by NGS sequencing techniques. The created genetic datasets are typically on the order of terabytes, and effectively and efficiently analyzing that amount of data is both procedurally and computationally demanding. For instance, analyzing NGS sequencing involves several baseline processing steps such as, e.g., aligning reads to one another, aligning and mapping reads to a reference genome, identifying and calling variant genes, identifying and calling abnormally methylated genes, generating functional annotations, etc. Performing any of these processes on terabytes of genetic data is computationally expensive for even the most powerful of computer architectures, and completely impossible for a normal human mind. Additionally, with the genetic sequencing data derived from the error-prone processes of sample preparation and sequence reading, large portions of the resulting genetic data may be low-quality or unusable for cancer identification. For example, large amounts of the genetic data may include contaminated samples, transcription errors, mismatched regions, overrepresented regions, etc. and may be unsuitable for high accuracy cancer detection. Identifying and accounting for low quality genetic data across the vast amount of genetic data obtained from NGS sequencing is also procedurally and computationally rigorous to accomplish and is also not practically performable by a human mind. Overall, any process created that leads to more efficient processing of large array sequencing data would be an improvement to cancer detection using NGS sequencing.

Finally, and perhaps most importantly, accurate identification of informative DNA from NGS data to identify a cancer presence is also difficult (much more in the early cancer detection context). To be effective, algorithms are sought to compensate for, e.g., errors generated by sample preparation and sequencing, and to overcome the large-scale data analysis problems accompanying NGS techniques. That is, designing a machine learning model or models, or other computational processing algorithms, that enable early cancer detection based on next generation sequencing techniques must be configured to account for the problems that those techniques create. Some of those techniques and models are discussed hereinbelow and particular improvements to state-of-the-art techniques and models are further discussed.

The present disclosure addresses some of the problems identified above by providing improved systems and methods for sample contamination detection of contaminated fragments for cancer classification. The system utilizes substantially unique sample barcode sequences that are ligated to nucleic acid (NA) molecules originating from one sample. The sample barcode sequences in addition to the one or more indices in a sequencing library help to accurately assign sequence reads to the appropriate sample, thereby providing an improvement to next-generation sequencing technology and, more generally, to the physical assaying process. The ligation of the sample barcodes upstream of multiplex sequencing provides a foundation for increased multiplexing, i.e., increased number of samples sequenced together in one flow cell. In particular, the added layer of contamination detection with the sample barcode can identify and detect the higher frequency of contamination (e.g., index hopping events), to guard against skewed analyses from contaminated samples.

Upon detection of a contaminated sample, the analytics system can perform remedial measures. For example, the analytics system may remove a contaminated sample from use in training of the cancer classification model, thereby improving the accuracy and precision of the cancer classification model. To expand further, the analytics system may perform contamination detection from an initial set of training samples to determine any contaminated samples to remove form the set of training samples. The filtered set of training samples (free of contamination) may be used for training of models. As another example, the analytics system may withhold a contaminated test sample from cancer classification, thereby reducing the false-positive rate of prediction of cancer.

Upon detection of a contaminated sample, further remedial measures may be taken to address the contamination. Other remedial measures may include identifying and remedying a source of the contamination through performing tests on varying conditions of the sample preparation process to pinpoint conditions that contribute to the contamination. Remedying sources of contamination thereby improves the physical assaying process and the sample processing. Upon identifying the source of contamination, action be undertaken to remove the contamination source, or minimize the contamination in the sample processing workflow. Another remedial measure may be physically discarding the contaminated sample, and optionally obtaining a new sample for the individual.

The training of the machine-learned models described herein (such as the cancer classifier, neural networks, and other models referenced herein) include the performance of one or more non-mathematical operations or implementation of non-mathematical functions at least in part by a machine or computing system, examples of which include but are not limited to data loading operations, data storage operations, data toggling or modification operations, non-transitory computer-readable storage medium modification operations, metadata removal or data cleansing operations, data compression operations, protein structure modification operations, image modification operations, noise application operations, noise removal operations, and the like. Accordingly, the training of the machine-learned models described herein may be based on or may involve mathematical concepts, but is not simply limited to the performance of a mathematical calculation, a mathematical operation, or an act of calculating a variable or number using mathematical methods.

Likewise, it should be noted that the training of these models described herein cannot be practically performed in the human mind. The models are innately complex including vast amounts of weights and parameters associated through one or more complex functions. Training and/or deployment of such models involve so great a number of operations that it is not feasibly performable by the human mind alone, nor with the assistance of pen and paper. In such embodiments, the operations may number in the hundreds, thousands, tens of thousands, hundreds of thousands, millions, billions, or trillions. Moreover, the training data may include hundreds, thousands, tens of thousands, hundreds of thousands, millions, or billions of sequence reads, each sequence read may further include anywhere from hundreds up to thousands of nucleotides. Accordingly, such models are necessarily rooted in computer-technology for their implementation and use.

### I.A. Overview of Cancer Classification Workflow

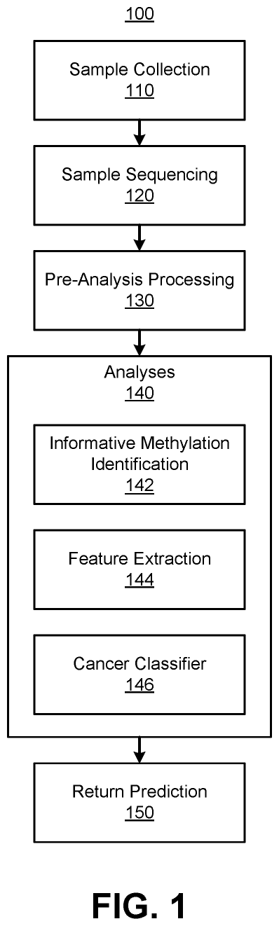

FIG. 1 is an exemplary flowchart describing an overall workflow 100 of cancer classification of a sample, according to one or more embodiments. The workflow 100 is by one or more entities, e.g., including a healthcare provider, a sequencing device, an analytics system, etc. Objectives of the workflow include detecting and/or monitoring cancer in individuals. From a healthcare standpoint, the workflow 100 can serve to supplement other existing cancer diagnostic tools. The workflow 100 may serve to provide early cancer detection and/or routine cancer monitoring to better inform treatment plans for individuals diagnosed with cancer. The overall workflow 100 may include additional/fewer steps than those shown in FIG. 1.

A healthcare provider performs sample collection 110. An individual to undergo cancer classification visits their healthcare provider. The healthcare provider collects the sample for performing cancer classification. Examples of biological samples include, but are not limited to, tissue biopsy, blood, whole blood, plasma, serum, urine, cerebrospinal fluid, fecal, saliva, sweat, tears, pleural fluid, pericardial fluid, or peritoneal fluid of the subject. The sample includes genetic material belonging to the individual, which may be extracted and sequenced for cancer classification. Once the sample is collected, the sample is provided to a sequencing device. Along with the sample, the healthcare provider may collect other information relating to the individual, e.g., biological sex, age, ethnicity, smoking status, any prior diagnoses, etc.

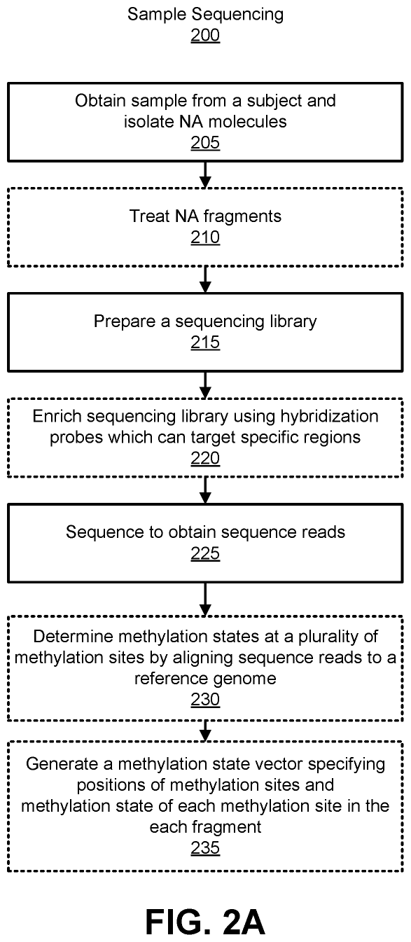

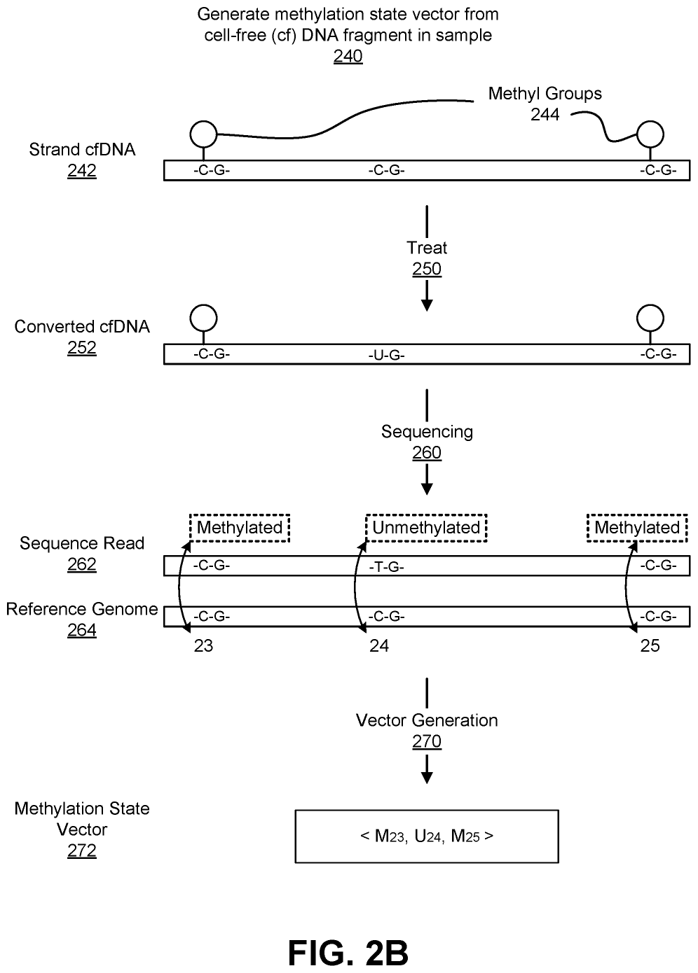

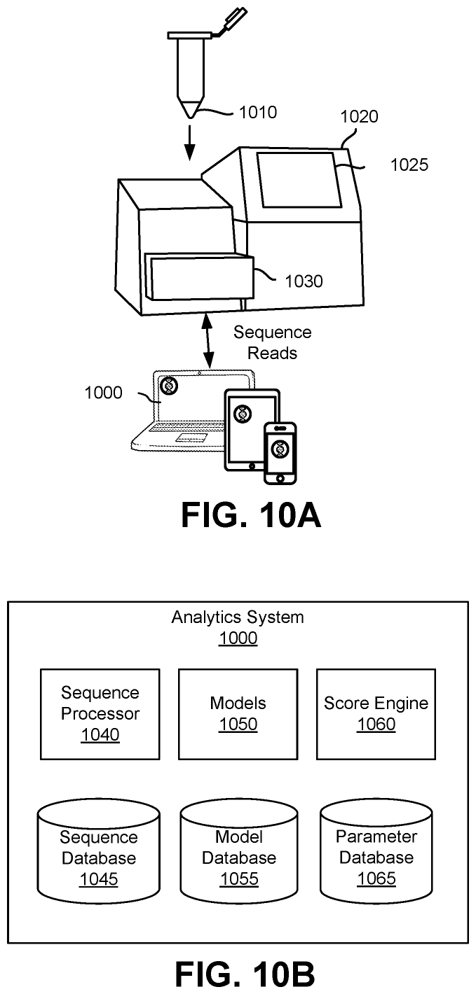

A sequencing device performs sample sequencing 120. A lab clinician may perform one or more processing steps to the sample in preparation of sequencing. Once prepared, the clinician loads the sample in the sequencing device. An example of devices utilizes in sequencing is further described in conjunction with FIGS. 10A & 10B. The sequencing device generally extracts and isolates fragments of nucleic acid that are sequenced to determine a sequence of nucleobases corresponding to the fragments. Sample sequencing includes sample treatment in preparation for sequencing of the fragments in the sample. Sample treatment may include one or more ligation steps, and amplification of the nucleic acid material. In one or more embodiments, the sample treatment includes ligation of a sample barcode and unique molecule identifiers that may be utilized in contamination detection. The sample barcode is a polynucleotide sequence that is substantially unique to each sample. The sample barcode is ligated onto each fragment in a sample prior to indexing and sequencing. The unique molecule identifiers are also polynucleotide sequences that are ligated onto each fragment originating in the sample, e.g., prior to amplification. The unique molecule identifiers may be utilized in de-duping sequence reads to identify unique fragments originating in the sample. Further description of sample sequencing 120 is described in FIGS. 2-7.

Different sequencing processes include Sanger sequencing, fragment analysis, and next-generation sequencing. Sequencing may be whole-genome sequencing or targeted sequencing with a target panel. In context of DNA methylation, bisulfite sequencing (e.g., further described in FIGS. 2A, & 2B) can determine methylations status through bisulfite conversion of unmethylated cytosines at CpG sites. Sample sequencing 120 yields sequences for a plurality of nucleic acid fragments in the sample. In one or more embodiments, the sequences may include methylation state vectors, wherein each methylation state vector describes the methylation statuses for CpG sites on a fragment.

An analytics system performs pre-analysis processing 130. An example analytics system is described in FIG. 10B. Pre-analysis processing 130 may include, but not limited to, demultiplexing, de-duplication of sequence reads, determining metrics relating to coverage, identification of contamination events, determining whether the sample is contaminated, remedial measures to contamination events, calling sequencing error, performing remedial measures, etc. As a result of the pre-analysis processing 130, the analytics system collects a set of sequence reads pertaining to the sample usable for the analyses 140.

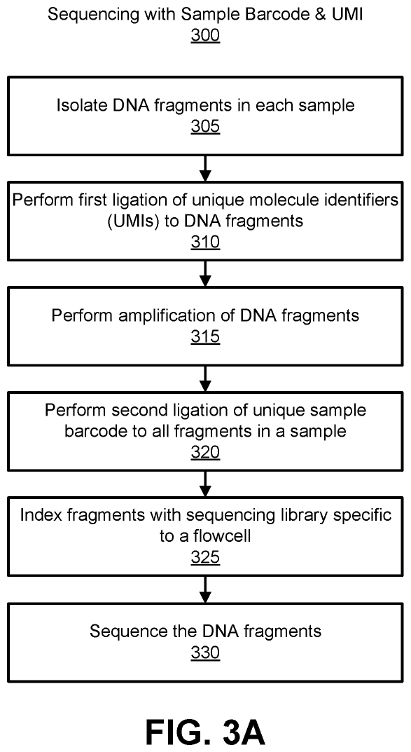

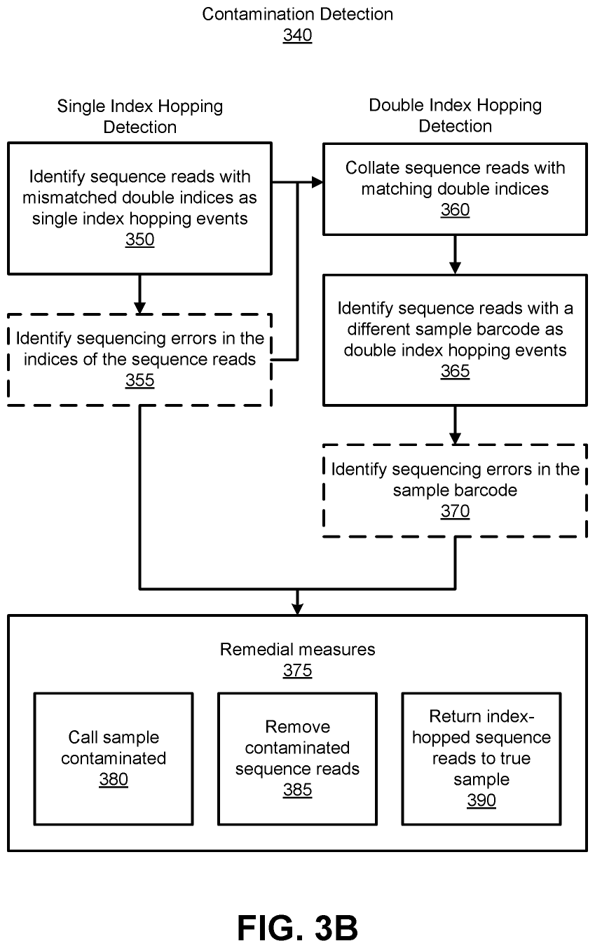

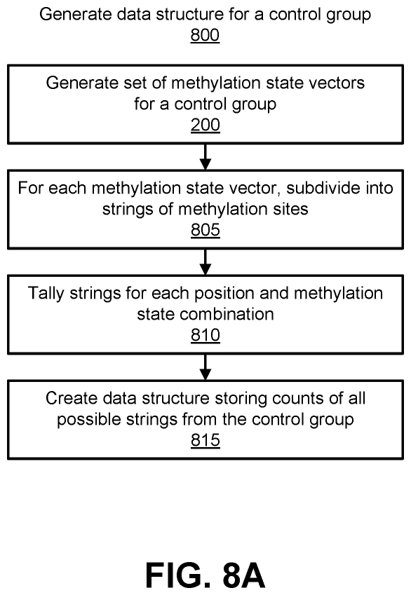

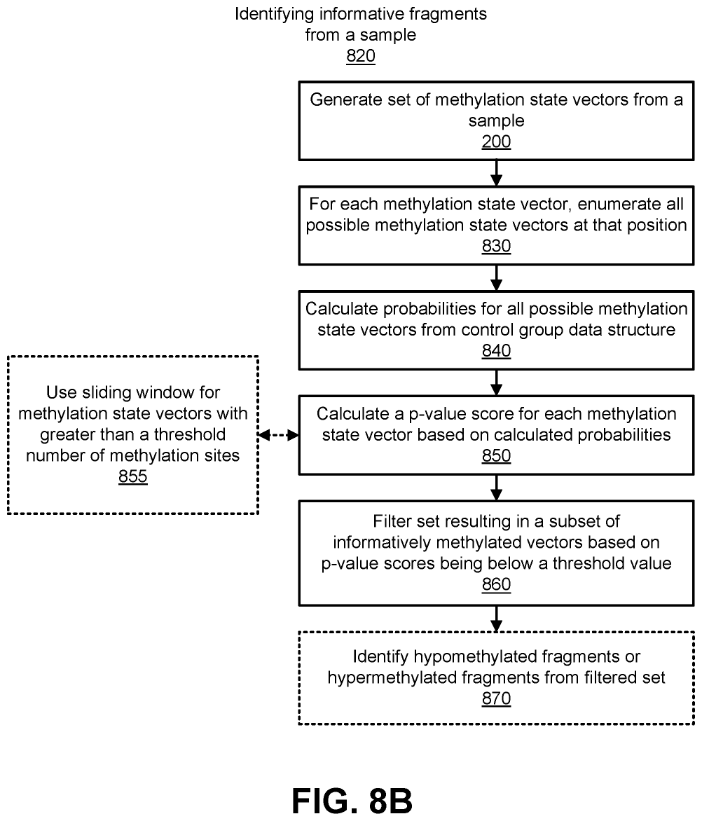

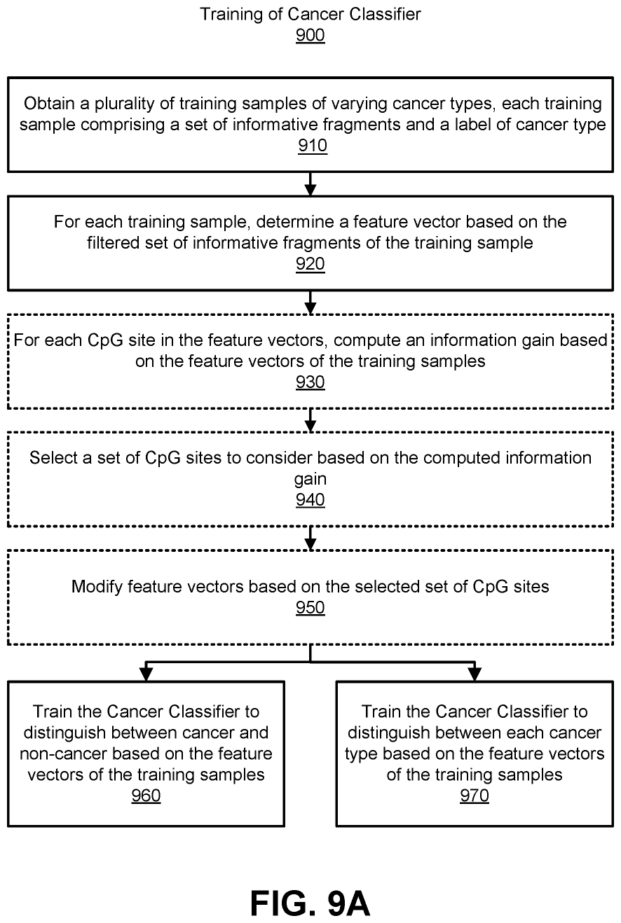

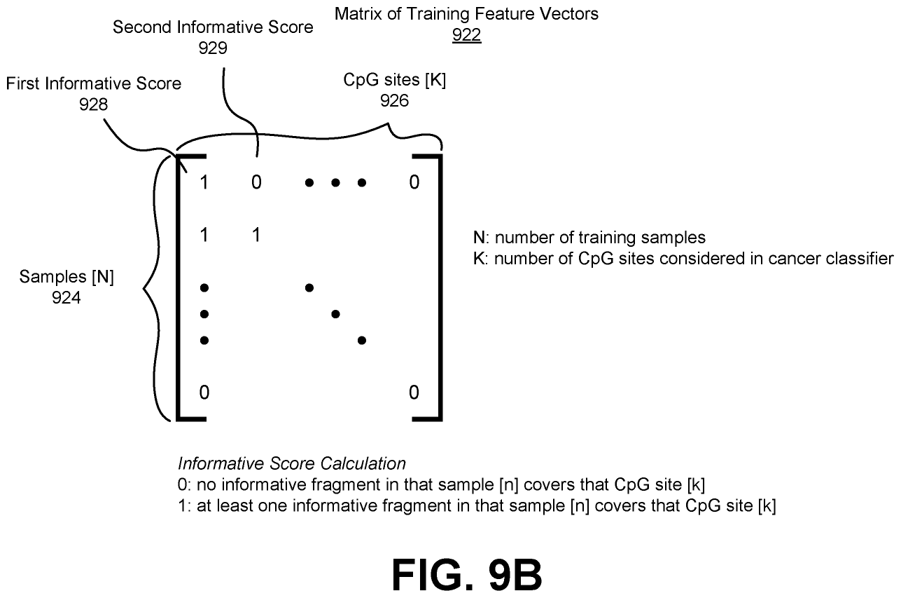

The analytics system performs one or more analyses 140. The analyses are statistical analyses or application of one or more trained models to predict at least a cancer status of the individual from whom the sample is derived. Different genetic features may be evaluated and considered, such as methylation of CpG sites, single nucleotide polymorphisms (SNPs), insertions or deletions (indels), other types of genetic mutation, etc. Contamination detection, as one example analysis, is further described in FIGS. 3A-7. In context of methylation, analyses 140 may include informative methylation identification 142 (e.g., further described in FIGS. 8A & 8B), feature extraction 144 (e.g., further described in FIGS. 9A & 9B), and applying a cancer classifier 146 to determine a cancer prediction (e.g., further described in FIG. 9A & 9B). In one or more embodiments of feature extraction, the analytics system may utilize one or more age covariate prediction models to generate one or more age covariate residuals as features to cancer classification. The cancer classifier 146 inputs the extracted features to determine a cancer prediction. The cancer prediction may be a label or a value. The label may indicate a particular cancer state, e.g., binary labels can indicate presence or absence of cancer, multiclass labels can indicate one or more cancer types from a plurality of cancer types that are screened for. The value may indicate a likelihood of a particular cancer state, e.g., a likelihood of cancer, and/or a likelihood of a particular cancer type.

The analytics system returns the prediction 150 to the healthcare provider. The healthcare provider may establish or adjust a treatment plan based on the cancer prediction. Optimization of treatment is further described in Section IV.C. Treatment.

### I.B. Overview of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of informatively methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of informatively methylated cfDNA fragments. First off, determining a DNA fragment to be informatively methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be informatively methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Informative DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.C. Definitions

The term “cell-free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “NA fragment,” or “NA molecule” may generally refer to any nucleic acid molecule, including DNA molecules and ribonucleic acid (RNA) molecules.

The term “amplicon” may generally refer to nucleic acid molecules resulting from an amplification process, i.e., including molecules originating from a sample taken from an individual and/or synthetically generated molecules as copies of original molecules.

The term “sample barcode” may generally refer to a nucleotide sequence that is assigned to a sample and ligated onto sequence reads, for the purpose of accurate assignment of sequence reads as belonging to the sample.

The term “molecule identifier,” or “MI” may generally refer to a nucleotide

sequence that is ligated onto original NA molecules originating from a sample, for the purpose of identifying distinct original NA molecules. The term “unique molecule identifier,” or “UMI” generally refers to a molecule identifier that is substantially unique compared to other UMIs.

The term “contamination event” generally refers to an instance of contamination in a sample. Contamination events may include, but are not limited, single-index hopping events, double-index hopping events, sequencing errors, sample swap events, etc. A “single-index hopping event” may refer to an instance where a single index is different than anticipated. A “double-index hopping event” may refer to an instance where both indices are different than anticipated. A “sequencing error” may refer to an instance where one or more nucleotides are different in one amplicon compared to another amplicon due to an error in the sequencing process, which may include errors introduced in ligation, amplification, etc. A “sample swap event” may refer to an instance where a sample is mislabeled as belonging to another sample.

The term “informative fragment,” “informatively methylated fragment,” or “fragment with an informative methylation pattern” refers to a fragment that has anomalous methylation of CpG sites compared to methylation statuses from a population of healthy subjects. Anomalous methylation of a fragment may be determined using probabilistic models to identify unexpectedness of observing a fragment's methylation pattern in the control group of healthy subjects.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “informative score” refers to a score for a CpG site based on a number of informative fragments (or, in some embodiments, UFXMs) from a sample overlaps that CpG site. The informative score is used in context of featurization of a sample for classification.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Informative cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes.

Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 450 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “bag” refers to a manner of grouping sequence reads together. For example, in demultiplexing, bags may be used to separate sequence reads as belonging to particular samples. As another example, in de-duping, bags may be used to identify sequence reads pertaining to amplicons of the same original DNA fragment in a sample.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### I.D. Example Analytics System

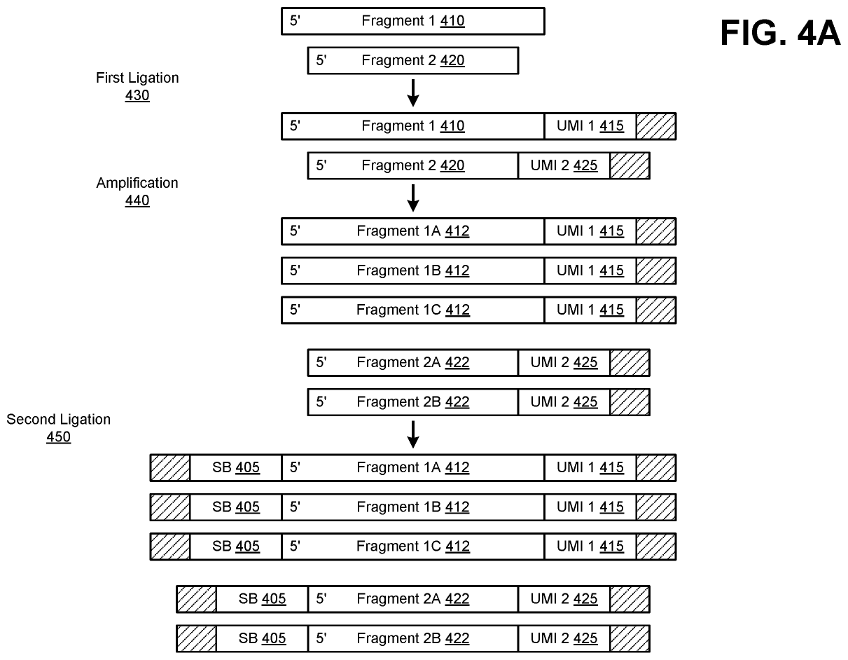

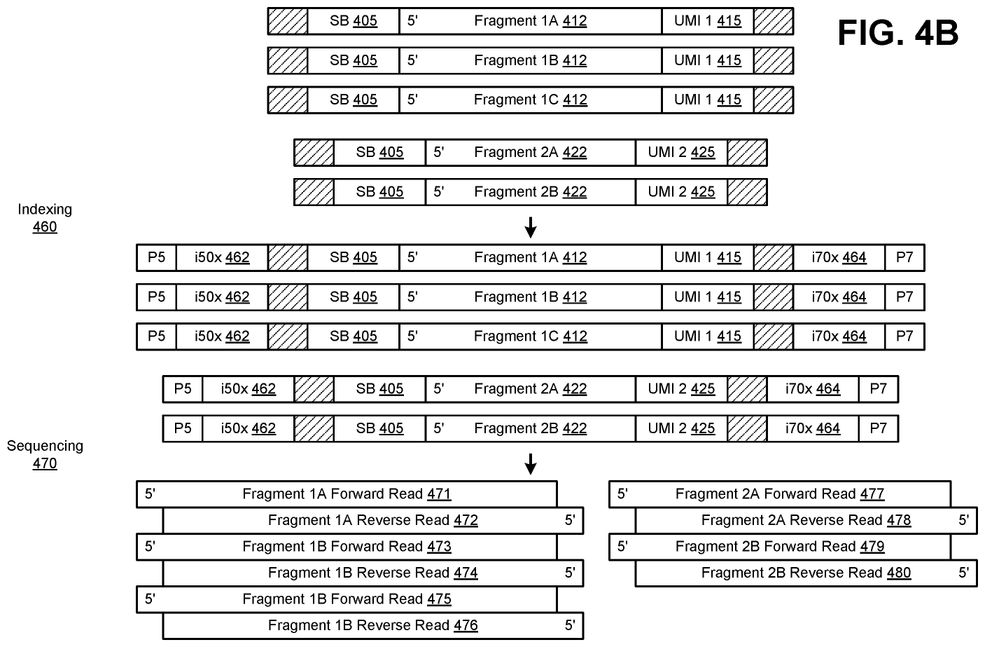

FIG. 10A is an exemplary flowchart of devices for sequencing nucleic acid samples according to one or more embodiments. This illustrative flowchart includes devices such as a sequencer 1020 and an analytics system 1000. The sequencer 1020 and the analytics system 1000 may work in tandem to perform one or more steps in the processes 300 of FIG. 3A, 400 of FIG. 4A, 420 of FIG. 4B, and other process described herein.

In various embodiments, the sequencer 1020 receives an enriched nucleic acid sample 1010. As shown in FIG. 10A, the sequencer 1020 can include a graphical user interface 1025 that enables user interactions with particular tasks (e.g., initiate sequencing or terminate sequencing) as well as one more loading stations 1030 for loading a sequencing cartridge including the enriched fragment samples and/or for loading necessary buffers for performing the sequencing assays. Therefore, once a user of the sequencer 1020 has provided the necessary reagents and sequencing cartridge to the loading station 1030 of the sequencer 1020, the user can initiate sequencing by interacting with the graphical user interface 1025 of the sequencer 1020. Once initiated, the sequencer 1020 performs the sequencing and outputs the sequence reads of the enriched fragments from the nucleic acid sample 1010.

In some embodiments, the sequencer 1020 is communicatively coupled with the analytics system 1000. The analytics system 1000 includes some number of computing devices used for processing the sequence reads for various applications such as assessing methylation status at one or more CpG sites, variant calling or quality control. The sequencer 1020 may provide the sequence reads in a BAM file format to the analytics system 1000. The analytics system 1000 can be communicatively coupled to the sequencer 1020 through a wireless, wired, or a combination of wireless and wired communication technologies. Generally, the analytics system 1000 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information, e.g., via step 340 of the process 300 in FIG. 3A. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 1000 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 10B, FIG. 10B is a block diagram of an analytics system 1000 for processing DNA samples according to one embodiment. The analytics system implements one or more computing devices for use in analyzing DNA samples. The analytics system 1000 includes a sequence processor 1040, sequence database 1045, model database 1055, models 1050, parameter database 1065, and score engine 1060. In some embodiments, the analytics system 1000 performs some or all of the processes 300 of FIG. 3A and 400 of FIG. 4A.

The sequence processor 1040 generates methylation state vectors for fragments from a sample. At each CpG site on a fragment, the sequence processor 1040 generates a methylation state vector for each fragment specifying a location of the fragment in the reference genome, a number of CpG sites in the fragment, and the methylation state of each CpG site in the fragment whether methylated, unmethylated, or indeterminate via the process 300 of FIG. 3A. The sequence processor 1040 may store methylation state vectors for fragments in the sequence database 1045. Data in the sequence database 1045 may be organized such that the methylation state vectors from a sample are associated to one another.

Further, multiple different models 1050 may be stored in the model database 1055 or retrieved for use with test samples. In one example, a model is a trained cancer classifier for determining a cancer prediction for a test sample using a feature vector derived from informative fragments. The training and use of the cancer classifier will be further discussed in conjunction with Section III. Cancer Classifier for Determining Cancer. The analytics system 1000 may train the one or more models 1050 and store various trained parameters in the parameter database 1065. The analytics system 1000 stores the models 1050 along with functions in the model database 1055.

During inference, the score engine 1060 uses the one or more models 1050 to return outputs. The score engine 1060 accesses the models 1050 in the model database 1055 along with trained parameters from the parameter database 1065. According to each model, the score engine receives an appropriate input for the model and calculates an output based on the received input, the parameters, and a function of each model relating the input and the output. In some use cases, the score engine 1060 further calculates metrics correlating to a confidence in the calculated outputs from the model. In other use cases, the score engine 1060 calculates other intermediary values for use in the model.

### II. Sample Sequencing & Processing

### II.A. Generating Methylation State Vectors for DNA Fragments

FIG. 2A is an exemplary flowchart describing a process 200 of sequencing a sample comprising nucleic acid (NA) fragment, according to one or more embodiments. In order to analyze NA methylation, an analytics system first obtains 205 a sample from an individual comprising a plurality of NA molecules. The process 200 may be applied to sequence many different types of NA molecules, e.g., DNA molecules, RNA molecules, cell-free DNA molecules, circulating-tumor DNA molecules, tissue DNA molecules, other types of NA molecules, etc. The process 200 is an embodiment of sample sequencing 120 of FIG. 1.

Generally, the sample sequencing process 200 comprises at least three steps. The analytics system obtains 205 a sample from a subject comprising NA molecules and isolates the NA molecules. The sample may be any type of biological sample originating from an individual, which comprises NA molecules. For example, the sample could be a blood sample, a urine sample, a tissue sample, another type of biological sample, etc. The analytics system prepares 215 a sequencing library that prepares the NA molecules for sequencing. Sequencing library preparation may include one or more ligation steps to add additional molecules used in the sequencing of the NA molecules, amplification of the NA molecules to create amplified molecules to ensure capture and sequencing of all NA molecules in the sample, enriching the sample by targeting specific genomic regions with targeting probes, ligation of one or more indices and one or more adaptors onto the NA molecules. Then the analytics system sequences 225 the NA molecules to obtain sequence reads. The sequence reads may include forward reads and reverse reads.

In one or more embodiments of methylation sequencing, the analytics system obtains 205 the sample comprising DNA fragments (e.g., cfDNA) and isolates each DNA fragment. The DNA fragments can be treated 210 prior to the sequencing. to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, CA)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, MA).

From the converted DNA fragments, a sequencing library can be prepared 215. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 220 for DNA fragments, or genomic regions, that are informative for cancer status using a plurality of hybridization probes. The hybridization probes are short oligonucleotides capable of hybridizing to particularly specified DNA fragments, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfite) for conversion of unmethylated cytosines to uracils. During enrichment, hybridization probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10 s, 100 s, or 1000 s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced 225 to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

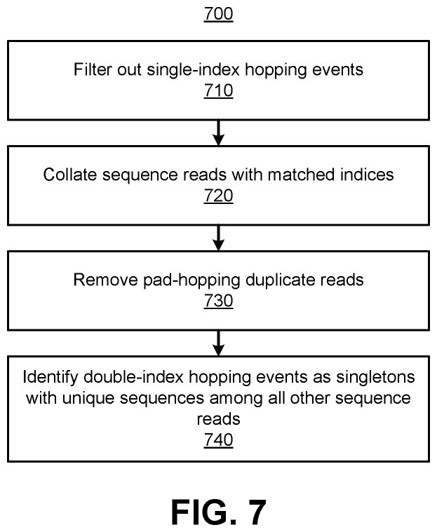

From the sequence reads, the analytics system determines 230 a location and methylation state for each CpG site based on alignment to a reference genome. The analytics system generates 235 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses. FIG. 7 further illustrates the process 200 in methylation sequencing embodiments.

### II.B. Methylation Sequencing

FIG. 2B is an exemplary illustration of methylation sequencing a cfDNA molecule to obtain a methylation state vector, according to one or more embodiments. As an example, the analytics system receives a cfDNA molecule 242 that, in this example, contains three CpG sites. As shown, the first and third CpG sites of the cfDNA molecule 242 are methylated 244. During the treatment step 250, the cfDNA molecule 242 is converted to generate a converted cfDNA molecule 252. During the treatment 250, the second CpG site which was unmethylated has its cytosine converted to uracil. However, the first and third CpG sites were not converted.

After conversion, a sequencing library is prepared and the molecule sequenced 260 to generate a sequence read 262. The analytics system aligns the sequence read 262 to a reference genome 264. The reference genome 264 provides the context as to what position in a human genome the fragment cfDNA originates from. In this simplified example, the analytics system aligns 270 the sequence read 262 such that the three CpG sites correlate to CpG sites 23, 24, and 25 (arbitrary reference identifiers used for convenience of description). The analytics system can thus generate information both on methylation status of all CpG sites on the cfDNA molecule 242 and the position in the human genome that the CpG sites map to. As shown, the CpG sites on sequence read 262 which are methylated are read as cytosines. In this example, the cytosines appear in the sequence read 262 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 270 a methylation state vector 272 for the fragment cfDNA 242. In this example, the resulting methylation state vector 272 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 4500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing.

For further details regarding methylation sequencing (e.g., WGBS and/or targeted methylation sequencing), see, e.g., U.S. patent application Ser. No. 16/352,602, entitled “Methylation Fragment Anomaly Detection,” filed Mar. 13, 2019, and U.S. patent application Ser. No. 16/719,902, entitled “Systems and Methods for Estimating Cell Source Fractions Using Methylation Information,” filed Dec. 18, 2019, each of which is hereby incorporated by reference. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments across each corresponding plurality of nucleic acid methylation fragments can comprise 1000 or more nucleic acid methylation fragments, 5000 or more nucleic acid methylation fragments, 10,000 or more nucleic acid methylation fragments, 20,000 or more nucleic acid methylation fragments, or 30,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 480 nucleotides.

Further details regarding methods for sequencing nucleic acids and methylation sequencing data are disclosed in U.S. patent application Ser. No. 17/191,914, titled “Systems and Methods for Cancer Condition Determination Using Autoencoders,” filed Mar. 4, 2021, which is hereby incorporated herein by reference in its entirety.

### II.C. Sample Contamination Detection With Sample Barcode

FIGS. 3A & 3B illustrate flowcharts for utilizing sample barcodes and unique molecule identifiers (UMIs) to detect contamination events, according to one or more embodiments. In particular, FIG. 3A illustrates an exemplary flowchart for DNA fragment isolation, sequencing library preparation, and DNA fragment sequencing, and FIG. 3B illustrates an exemplary flowchart of detecting contamination events from the sequence reads based on the sample barcode and the UMI. In one or more embodiments, a sequencer (e.g., the sequencer 1020 of FIG. 10) performs the process 300 in FIG. 3A, while the analytics system performs the process 335 of FIG. 3B. In other embodiments, any other combination of devices shown in FIGS. 10A & 10B may perform the steps shown in FIGS. 3A & 3B. In additional embodiments, some of the steps may be performed by a laboratory technician or scientist, e.g., ligations, amplification, indexing with sequence libraries, etc.

FIG. 3A illustrates the exemplary process 300 of sequencing DNA fragments with the sample barcode and UMIs. The sequencer isolates 305 DNA fragments in each sample of a plurality of samples. The sequencer (or a clinician) may perform a series of steps in isolation of the DNA fragments. For example, DNA isolation may involve lysing the sample, clearing of the lysate, binding via a matrix to isolate DNA fragments, washing of other contaminants, etc. Additional steps unique to each type of sample may be implemented. For example, plasma separation may be utilized with blood samples.

The sequencer performs 310 a first ligation of UMIs to the isolated DNA fragments. At this step, the DNA fragments present in the sample are all original molecules originating from the individual. A set of UMIs are utilized. Each UMI is a polynucleotide sequence that is substantially unique from the other UMIs in the set. The UMI length may be 4, 5, 6, 7, 8, 9, 10, 11, 12, 13, 14, 15, 16, 17, 18, 19, or 20 basepairs. In one embodiment, all UMIs in the set are the same length. In other embodiments, at least two UMIs are of differing lengths. Substantial uniqueness indicates some threshold difference between two UMIs. The threshold difference may be at least two different nucleotides, at least three different nucleotides, etc. Difference between UMIs can be based on a variety of metrics, e.g., nucleotides, length of the nucleotides, diversity of the nucleotide sequence, etc. The UMIs may be ligated onto one of the two ends of the DNA fragments. For example, the UMIs are ligated to the 3′ end, or, in the alternative, the UMIs are ligated to the 5′ end. In one or more embodiments, a ligase is added to the reaction to aid in the first ligation of the UMIs to the DNA fragments.

The number of UMIs in the set of UMIs may be tailored to optimize between collisions and false positive distinctive fragments. A collision occurs when multiple fragments bind to the multiple copies of the same UMI. A false positive distinctive fragment occurs when a sequencing error in the UMI for one of two sequence reads for the same fragment cause the sequencer to consider the two sequence reads as distinctive fragments. A smaller set of UMIs reduces rate of false positive distinctive fragments but potentially increases collision rate. A larger set of UMIs reduces collision rate but potentially increases rate of false positive distinctive fragments. The size of the set of UMIs may be 5, 10, 15, 20, 25, 30, 35, 40, 45, 50, 55, 60, 65, 70, 75, 80, 85, 90, 95, or 100.

The sequencer performs 315 amplification of the DNA fragments appended with the UMIs. Amplification results in multiple copies of each DNA fragment. These copies are synthetic, i.e., not originating from the sample, but based on DNA fragments in the sample. In one or more embodiments, amplification may be accomplished through polymerase chain reaction (PCR) amplification, linear amplification, clonal amplification, isothermal amplification, loop mediated isothermal amplification, nucleic acid sequence based amplification, strand displacement amplification, rolling circle amplification, ligase chain reaction, ramification amplification, other types of amplification techniques, etc. Each of the copies (“amplicons”) generally includes the UMI sequence appended to each original DNA fragment (unless amplification error affects the UMI sequence during the amplification).

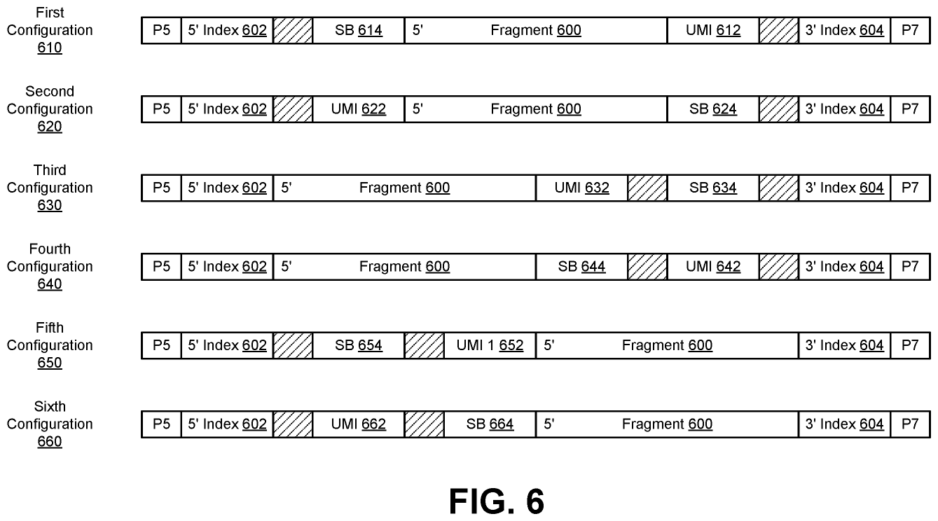

The sequencer performs 320 a second ligation of a unique sample barcode to all fragments in one sample. As noted, the amplicons presumably include the UMI sequence appended in the first ligation to the original DNA fragments. The second ligation includes ligation of a unique sample barcode to all amplicons belonging to the sample. The second ligation occurring after amplification reduces chances of amplification error affecting the sample barcode sequence appended to the amplicons. A sample barcode is a polynucleotide sequence. The sample barcodes may have a length of 4, 5, 6, 7, 8, 9, 10, 11, 12, 13, 14, 15, 16, 17, 18, 19, or 20 basepairs. All amplicons for one sample are ligated with the same sample barcode. Each sample barcode is substantially unique to the other sample barcodes. Substantially unique means some threshold difference between any two sample barcodes. Difference can be based on different nucleotide sequences, length of nucleotide sequences, diversity of nucleotide sequences, etc. The sample barcode may be ligated onto one of the two ends of the DNA fragments. For example, the sample barcodes are ligated to the 3′ end, or, in the alternative, the sample barcodes are ligated to the 5′ end. In one or more embodiments, a ligase is added to the reaction to aid in the first ligation of the UMIs to the DNA fragments. Configurations of the UMIs and the sample barcodes are further illustrated in FIG. 6.

The sequencer indices 325 the fragments with a sequencing library specific to a well of the flow cell in the sequencer. In one or more embodiments, the sequencer is configured for multiplex sequencing, e.g., each flow cell contains multiple wells, wherein each well is configured to sequence DNA fragments belonging to one sample. The sequencer's sequencing library comprises indices specified for each well in the flow cell. The indices are polynucleotide sequences used by the sequencer to anneal primers for a DNA polymerase to add fluorescent deoxyribose nucleotide triphosphate (dNTPs) that are used to sequence the DNA fragments. The indices may have a length of 4, 5, 6, 7, 8, 9, 10, 11, 12, 13, 14, 15, 16, 17, 18, 19, or 20 basepairs. In some embodiments adapter sequences are included on the indices, wherein the adapter sequences are polynucleotides that bind to oligos covering the flow cell. In one or more embodiments, two unique indices are utilized for each cell in the flow cell. One index is ligated to one end of the amplicons with UMI and sample barcode appended, while the other index is ligated to the opposite end of the amplicons. In other embodiments, a single index is utilized for each cell. The single index may be ligated to one of the ends, either the 3′ end or the 5′ end.

The sequencer sequences 330 the DNA fragments. At this step, the DNA fragments are amplicons with at least the sample barcode, the UMI, and one or more indices appended. The sequencer performs a series of steps to detect forward and reverse sequence reads for each of the amplicons.

In one example of sequencing by synthesis, DNA fragments are tethered to the flow cell, through a series of amplification steps (e.g., under step 315). While tethered, the DNA fragments are single stranded fragments. Reverse DNA fragments are cleaved leaving only the forward DNA fragments tethered to the flow cell. A first primer is introduced into the flow cell which anneal to the remaining forward DNA fragments. Fluorescent dNTPs are introduced into the flow cell, and DNA polymerases proceed with synthesizing complementary polynucleotides starting with the annealed primers. As dNTPs are added to the growing polynucleotide chain, light is used to excite the fluorescent tags which can be detected with an imaging device. Each fluorescent tag corresponds to a different nucleotide. This process continues until all forward sequence reads are obtained. The sequencer amplifies once more to obtain both forward DNA fragments and reverse DNA fragments tethered to the flow cell. The forward DNA fragments are cleaved leaving only the reverse DNA fragments. A similar process occurs where a second primer is introduced which anneals to the reverse DNA fragments. The DNA polymerase sequentially grows the chain of the reverse DNA fragments while exciting and fluorescing the fluorescent dNTPs, thereby obtaining the reverse sequence reads.

FIG. 3B is an exemplary flowchart of a process 340 for detecting contamination events from the sequence reads based on the sample barcode and the UMI. With the forward and reverse sequence reads, the analytics system identifies contamination events based on the sequence reads. The analytics system is capable of identifying, as contamination events, single index hopping events, double index hopping events, sequencing errors, or some combination thereof.

In single index hopping detection, the analytics system identifies 350 sequence reads with mismatched double indices as single index hopping events. For example, one uniquely-tagged library utilizes a first index for the 3′ end and a second index for the 5′ end, whereas another uniquely-tagged library utilizes a third index for the 3′ end and a fourth index for the 5′ end. If a sequence read includes nucleotide sequences corresponding to one index from the first library and another index from the second library, those are mismatched indices. For example, the sequence read includes nucleotide sequences corresponding to the first index and the fourth index, or the sequence read includes nucleotide sequences corresponding to the third index and the second index.

If the sequence read is not quite mismatched but not identical to the anticipated indices, then the analytics system may identify 355 one or more sequencing errors in the sequence reads. If, for example, a fragment has an index with one different nucleotide than the anticipated index sequence, whereas all indices have a distance of at least three (i.e., at least three different nucleotides), then the likelihood is that the single different nucleotide is due to sequencing error. In some embodiments, if the index sequence of the sequence read does not match exactly to any of the indices of the various libraries, then the analytics system may determine which index the index sequence in the sequence read is closest to. The analytics system may calculate a distance from each of the indices in the libraries. The distance may be based on number of nucleotide differences, e.g., the distance can be two if there are two nucleotides in the sequence read that are different from the index. The index with the smallest distance from the sequence read is deemed the closest index. The different nucleotides may be determined to be sequencing errors. In some embodiments, the closest index is determined to be the appropriate index to match with the other index.

The analytics system collates 360 sequence reads with matching double indices. The analytics system may include sequence reads with one or more different nucleotides determined to be sequencing error. For example, a first index is paired with a second index in a sequencing library. An example sequence read includes sequences for the second index and the first index with a determined sequencing error, then the sequence read is determined to include both the first index and the second index.

The analytics system identifies 365 sequence reads with a different sample barcode as double index hopping events. The analytics system compares the sample barcode of the sequence reads collated in step 360. The analytics system may know the sample barcode assigned with the pair of indices collated. The analytics system identifies any sample barcode different that the assigned sample barcode as a double index hopping event. For example, the analytics system anticipates sequence reads collated in step 360 as having a sample barcode with the sequence of <ATGCTACGC> (from 5′ end to 3′ end). If the analytic system identifies anything but the sample barcode sequence, the analytics system can determine that sequence read to be a double index hopping event. For example, a sequence read with sample barcode sequence <CACCGTAA> (from 5′ end to 3′ end) is deemed to be a double index hopping event.

The analytics system may identify 370 sequencing errors in the sample barcode. If the sample barcode in step 365 is determined to be different that the assigned sample barcode, the analytics system may consider if the different nucleotides are due to sequencing error of the proper sample barcode. Recalling this example, the analytics system anticipates sequence reads collated in step 360 as having a sample barcode with the sequence of <ATGCTACGC> (from 5′ end to 3′ end). The analytics system identifiers another sequence read with sample barcode sequence of <ATGCGACGC> (from 5′ end to 3′ end). The bolded and underlined nucleotide is different than the assigned sample barcode. In step 365, the analytics system may have determined the sequence read to be a double index hopping event. In step 370, the analytics system further evaluates whether the different nucleotide is due to sequencing error. In some embodiments, the analytics system allows for a certain tolerance of error, e.g., if no more than one nucleotide is different. In other embodiments, the analytics system may identify the closest sample barcode from the plurality of sample barcodes used for the samples. The analytics system may determine a distance between the sequence read's sample barcode from the sample barcodes used in the sequencing process (e.g., as described in FIG. 3A). Distance can be measured based on number of different nucleotides, e.g., four different nucleotides can be assigned a distance of four. The distance can also be based on other metrics including, but not limited to, length of the sequence, diversity of the nucleotides in the sequence, etc. The sample barcode used in the sequencing process with the smallest distance from the sequence read's sample barcode is determined to be the closest sample barcode.

The analytics system performs 375 remedial measures. At step 375, the analytics system has identified contamination events (e.g., single index hopping, double index hopping, sequencing errors, or some combination thereof). The analytics system may undertake one or more of the various measures.

In one example remedial measure, the analytics system may call 380 the sample as contaminated. To call the sample as contaminated, the analytics system may utilize one or more rules. For example, one rule may be based on a threshold for a total number of contamination events detected (including single index hopping, double index hopping, sequencing errors, or some combination thereof). Another one or more rules may be based on hitting a threshold number of a particular contamination event (single index hopping, double index hopping, or sequencing errors). The analytics system may utilize the rules in conjunction or disjointly (whenever any rule is satisfied, then the sample is determined to be contaminated). The analytics system may further normalize based on sample coverage, e.g., sequencing depth. The analytics system may notify a healthcare provider of the contaminated sample, e.g., to prompt collection of a new sample.

In another example remedial measure, the analytics system may remove 385 contaminated sequence reads. The analytics system may exclude the contaminated sequence reads and proceed with downstream analyses on remaining sequence reads not contaminated.

In another example remedial measure, the analytics system may return 390 index-hopped sequence reads to true sample. As the sample barcodes operates to identify which sample a sequence read belongs to, in spite of index hopping events, the sequence reads retain the sample barcode as an identifier to the sample they belong. As such, the analytics system may return the sequence reads to true sample. For example, a first sequence read is collated into a first bag based on the double index collation (step 360). The first bag corresponds to a first sample, but the first sequence read has a sample barcode assigned to a second sample. The analytics system determines the first sequence read to be a double index hopping event, and then may place the first sequence read with a second bag for the second sample.

Utilization of the sample barcode in the process 340 of contamination detection provides precision in identifying specific sequence reads as contaminated. In other contamination detection workflows, an analytics system is not apprised of which sequence reads are contaminated, rather determines generally that the sample is contaminated. In such workflows, the only available recourse is to collect a new sample. In contrast, the process 340 of contamination detection allows for the identification of particular sequence reads that are contaminated during the sequencing process (e.g., single index hopping, double index hopping, sequencing errors, etc.), expanding the recourses available beyond discarding the contaminated sample and collecting a new sample.

Moreover, utilization of the sample barcode in the process 340 of contamination detection can improve methyl variant allele fraction calculations. MVAF (methyl variant allele fraction) is an important metric for post-diagnostic products (e.g., MRD, prognosis, etc). Compared to deduping workflows based on shared endpoints of sequence reads, those workflows may perform over-deduping as different original cfDNA fragments may share similar endpoints. The collision rate increases with higher coverage. At a high rate of collisions, MVAF can be inaccurate because of the underestimation of total coverage and/or abnormal coverage in MVAF targets. In workflows without sample barcodes, singletons (bag size of 1, or fragments without duplicates) are removed from the downstream analysis. The rate of fragment recovery is different between abnormal fragments and background fragments due to probe design. The duplication rate or percent singletons varies by input level as well. The linearity and accuracy of MVAF could be limited by these limitations. Implementing sample barcodes improves the fragment recovery rate, thereby providing for more accurate MVAF calculations.

FIGS. 4A & 4B are exemplary illustrations showing the sequencing process 300 of FIG. 3A utilizing sample barcodes and unique molecule identifiers. In particular, FIG. 4A is an exemplary illustration of sample treatment, according to one or more embodiments, and FIG. 4B is an exemplary illustration of sample sequencing following sample treatment in FIG. 4A, according to one or more embodiments. By way of illustration, a sample comprises original fragments, fragment 1 410 and fragment 2 420.

First ligation 430 occurs to ligate unique molecule identifiers (UMIs) to each original fragment. As shown in FIG. 4A, UMI 1 415 is ligated onto fragment 1 410, and UMI 2 425 is ligated onto fragment 2 420. UMI 1 415 and UMI 2 425 are substantially distinct. The diagonal hatched boxes represent nucleotides on the non-ligated end of the UMIs. The diagonal hatched boxes may be primers for binding enzymes to the fragments. The UMIs are ligated onto the 3′ end of the original fragments.

Amplification 440 occurs to amplify the original fragments ligated with UMIs. The resulting amplicons may comprise synthetically generated fragments and/or the original fragments. For example, fragment 1 410 is copied resulting in three amplicons, fragment 1A 412, fragment 1B 412, and fragment 1C 412. Each of the amplicons of fragment 1 410 includes UMI 1 415 that was ligated onto the original fragment 1 410. Fragment 2 420 is amplified resulting in two amplicons, fragment 2A 422 and fragment 2B 422, each with UMI 2 425.

At second ligation 450, a sample barcode is appended to the amplicons. In particular, all amplicons receive sample barcode (SB) 405 at the 5′ end, which is opposite the UMIs. The SB 405 may also comprise one or more nucleotides at the non-ligating end to protect the sample barcode.

At indexing 460 (turning to FIG. 4B), the fragments including the SB 405 and the UMIs are indexed for sequencing. Indexing involves ligation of index primers on either end of the fragments. In this example, i50x 462 with P5 nucleotide sequence is ligated onto the 5′ end adjacent to the SB 405 (optionally with protective nucleotide sequence), and i70x 464 with P7 nucleotide sequence is ligated onto the 3′ end adjacent to the UMIs (optionally with protective nucleotide sequence). The P5 and P7 nucleotide sequences bind to complementary oligos on the sequencer flow cell, i.e., to tether the fragments to the flow cell.

At sequencing 470, the sequencer sequences the fragments to obtain forward and reverse sequence reads for each of the fragments. The sequence reads include the indices, the sample barcode, the fragment sequences, and the UMIs. The sequence reads may omit the P5 and P7 nucleotide sequences. As such: fragment 1A 412 yields fragment 1A forward read 471 and fragment 1A reverse read 472; fragment 1B 412 yields fragment 1B forward read 473 and fragment 1B reverse read 474; fragment 1C 412 yields fragment 1C forward read 475 and fragment 1C reverse read 476; fragment 2A 422 yields fragment 2A forward read 477 and fragment 2A reverse read 478; fragment 2B 422 yields fragment 2B forward read 479 and fragment 2B reverse read 480.

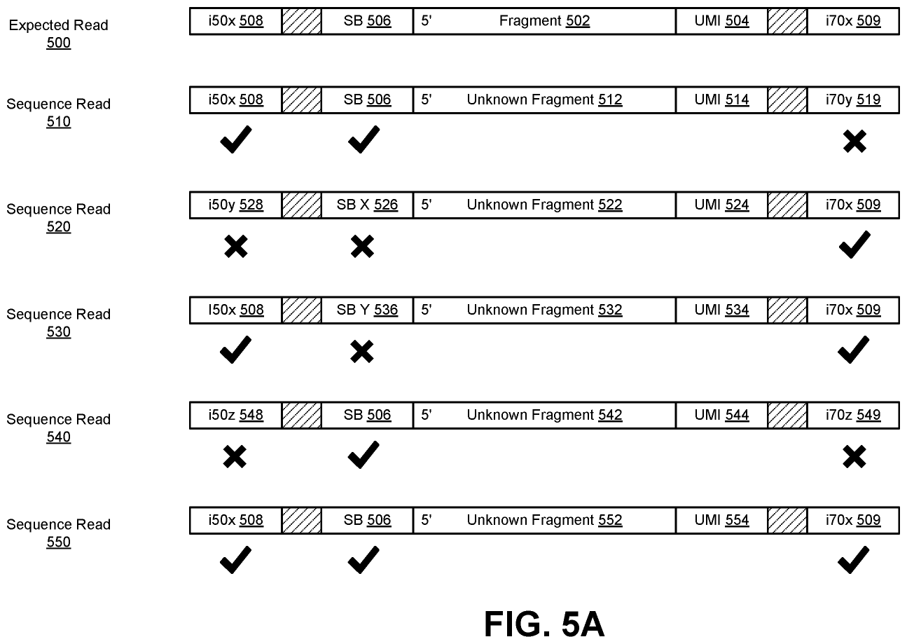

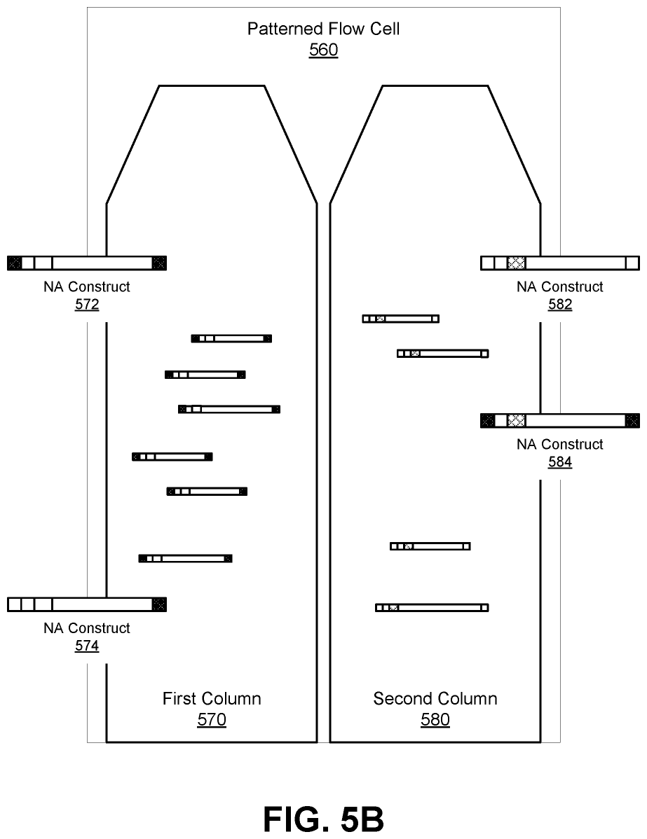

FIG. 5A is an exemplary illustration of contamination detection, according to one or more embodiments. FIG. 5A illustrates various sequence reads with different combinations of sample barcodes, UMIs, indices, etc. The sequence reads are used to identify contamination events (e.g., single index hopping, double index hopping, sequencing error, etc.). For example, the expected sequence read 500 (as a result of the sample treatment process as described and illustrated in FIGS. 3A, 4A, and 4B). The expected read 500 comprises, from 5′ end to 3′ end, a first index as i50x 508, a sample barcode (SB 506), the fragment sequence 502, a UMI 504, and a second index as i70x 509. In this example, the unique sequencing library utilizes index for 5′ end i50x 508 is used in conjunction with index for 3′ end i70x 509.

A first sequence read 510 has, from 5′ end to 3′ end, a first index as i50x 508, SB 506, unknown fragment sequence 512, UMI 514, and a second index as i70y 519. The first index of the first sequence read 510 (i50x 508) matches to the first index of the expected read 500 (150x 508). The second index of the first sequence read 510 (i70y 519) does not match to the second index of the expected read 500 (i70x 509). With mismatched indices (having two indices from two different sequencing libraries), the analytics system would determine the first sequence read 510 to be a single index hopping event. After determining the indices to be mismatched, the analytics system may proceed to evaluating the sample barcode. In this instance, the sample barcode matches to the anticipated sample barcode SB 506. In spite of the single index hopping event, the analytics system may determine the first sequence read 510 as belonging to the sample associated with the sample barcode SB 506. In another embodiment, the second index matches but the first index does not, still a mismatched pair. The analytics system may similarly determine the sequence read to be a single index hopping event.

A second sequence read 520 has, from 5′ end to 3′ end, a first index as i50y 528, SB X 526, unknown fragment sequence 522, UMI 524, and a second index as i70x 509. With the second sequence read 520, there is a mismatch in the paired of indices. The first index as i50y 528 is different than the first index of i50x 508 on the expected read 500, whereas the second index of i70x 509 matches to the second index of i70x 509 on the expected read 500. As with the first sequence read 510, the analytics system may determine that the mismatched indices for the second sequence read 520 is a single index hopping event. In the case of the second sequence read 520, the analytics system evaluates the sample barcode SB X 526. The analytics system determines that SB X 526 does not match to the assigned SB 506, and may assign the second sequence read as belonging to the sample associated with SB X 526. As a remedial measure, the analytics system may return the second sequence read 520 to the sample associated with the sample barcode SB X 526.

A third sequence read 530 comprises, from 5′ end to 3′ end, a first index as i50x 508, a sample barcode as SB Y 536, an unknown fragment sequence 532, UMI 534, and a second index as i70x 509. The analytics system evaluates the indices. Both indices match to the expected read 500. The indices also match to one another, i.e., the two indices are paired in one sequencing library. The analytics system next evaluates the sample barcode. The analytics system would determine the sample barcode of SB Y 536 is different than the SB 506. As such, the analytics system would determine the third sequence read 530 to be a double index hopping event. As a remedial measure, the analytics system may return the third sequence read 530 to the sample associated with the sample barcode of SB Y 536.

A fourth sequence read 540 comprises, from 5′ end to 3′ end, a first index as i50z 548, a sample barcode as SB 506, unknown fragment sequence 542, UMI 544, and i70z 549. The analytics system evaluates the indices. Both indices do not match to the expected read 500. The analytics system may have collated the fourth sequence read 540 with other sequence reads with the indices i50z 548 and i70z 549. The analytics system may determine the fourth sequence read 540 to be a double index hopping event. The analytics system then evaluates the sample barcode SB 506, which matches to the sample barcode of the expected read 500. In this case, the analytics system may return the fourth sequence read 540 to a bag of reads for the sample associated with the sample barcode SB 506.

A fifth sequence read 550 comprises, from 5′ end to 3′ end, a first index as i50x 508, SB 506, unknown fragment sequence 552, UMI 554, and a second index as i70x 509. The analytics system evaluates the indices. The fifth sequence read 550 has both indices matched to the expected read 500. The analytics system evaluates the sample barcode. With the fifth sequence read 550, the sample barcode of SB 506 matches to that of the expected read 500. The analytics system determines the fifth sequence read 550 to be free of contamination events.

The analytics system utilizes the UMI sequences in the sequence reads to identify unique original fragments. Each original fragment has a UMI ligated onto the fragment. The analytics system may identify original fragments based on at least the UMIs. In some embodiments, the analytics system may also base the identification on endpoints of the sequence reads, e.g., once aligned to a reference genome. For example, the analytics system may identify multiple sequence reads with the same UMI sequence and having similar endpoints as amplicons of the same original fragment. In another example, the analytics system identifies two sequence reads with differing UMI sequences as derived from two different original fragments. In yet another example, the analytics system identifies two sequence reads with same UMI sequences but different endpoints as two different original fragments. Utilizing UMIs and endpoint analysis decreases the likelihood of collision, i.e., two original fragments determined to be the same original fragment.

FIG. 5B is an exemplary illustration of nucleic acid constructs incorporating sample barcodes in a patterned flow cell 560, according to one or more embodiments. The patterned flow cell 560 is a slide that holds the samples when sequenced by the sequencer 1020. In one or more embodiments, the patterned flow cell 560 is a substrate with one or more columns in the substrate. Each column is separate from the other columns. Each column comprises a plurality of nanowells covered with a lawn of oligonucleotides that bind to complementary adaptors on the nucleic acid constructs (e.g., P5 & P7 as shown in other figures). One sequencing library, comprising one or two indices, is assigned to each sample sequenced on the patterned flow cell 560. In one or more embodiments, a single sample is assigned to each column. In the example of FIG. 5B, the patterned flow cell 560 is configured with two columns, the first column 570 and the second column 580. Two nucleic acid (NA) samples are prepared and multiplex sequenced in the two flow cells, e.g., according to the process 300 in FIG. 3A.

In the process, the analytics system may identify sequence reads belonging to each sample based on the index sequences and the sample barcode on the sequence reads.

Sequence reads belonging to the first sample sequenced in the first column 570 are anticipated as having double index sequences shown as the two black boxes on the nucleic acid (NA) constructs in the first column 570. Moreover, sequence reads belonging to the first sample are anticipated as having a white box as the sample barcode. As an example, NA construct 572 illustrates the anticipated double black box index sequences and the white box sample barcode sequence. The analytics system would determine the NA construct 572 to be free from contamination events. NA construct 574 is shown as having a mismatched double index sequence with the left outer box shown in white, but the NA construct 574 has the anticipated sample barcode sequence for the first sample. The analytics system would determine the NA construct 574 to have a single index hopping event, but the analytics system may assign the NA construct 574 to the first sample based on the appropriate sample barcode sequence.

Sequence reads belonging to the second sample sequenced in the second column 580 are anticipated as having double index sequences shown as the two white boxes on the nucleic acid (NA) constructs in the second column 580. Moreover, sequence reads belonging to the second sample are anticipated as having a hatched box as the sample barcode sequence. As another example, NA construct 582 sequenced in the second column 580 is free from contamination events. The NA construct 582 has the anticipated double index sequences and the anticipated sample barcode for the second sample. NA construct 584 has the double index sequence assigned to the first sample, the double black outer boxes, but with the sample barcode for the second sample. In the case of the NA construct 584, the analytics system would determine the NA construct 584 to be a double index hopping event.

FIG. 6 is an exemplary illustration of various configurations of sample barcodes and unique molecule identifiers, according to one or more embodiments. The various configurations utilize a fragment 600 that is to be sequenced with double-indexed libraries.

In a first configuration 610, a UMI 612 is ligated onto the 3′ end of the fragment 600 and a sample barcode SB 614 is ligated onto a 5′ end of the fragment 600. The two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

In a second configuration 620, a UMI 622 is ligated onto the 5′ end of the fragment 600 and a sample barcode SB 624 is ligated onto the 3′ end of the fragment 600. The two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

In the third, fourth, fifth, and sixth configurations, the UMI and the sample barcode are ligated onto the same side of the fragment.

In the third configuration 630, the UMI 632 is ligated onto the fragment 600 before the sample barcode SB 634 on the 3′ end of the fragment 600. Then the two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

In the fourth configuration 640, the sample barcode SB 644 is ligated onto the fragment 600 before the UMI 642 on the 3′ end of the fragment 600. Then the two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

In the fifth configuration 650, the UMI 652 is ligated before the sample barcode 654 on the 5′ end of the fragment 600. Then the two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

In the sixth configuration 660, the sample barcode 664 is ligated before the UMI 662 on the 5′ end of the fragment 600. Then the two indices are ligated after the sample barcode and UMI. In particular, a 5′ index 602 is ligated after the sample barcode 614, and a 3′ index 604 is ligated after the UMI 612. The fragment also has oligos P5 and P7 after the indices, with P5 ligated onto the 5′ end and P3 ligated onto the 3′ end.

### II.D. Additional Contamination Detection

In some embodiments, the analytics system is capable of identifying sequencing errors that arise in singleton sequence reads. A singleton is a read with a sequence that is present only once (unique/isolated) among the reads. A singleton has no PCR duplicates. Additionally, a singleton with UMI is a single sequence read with a UMI and no other PCR duplicates. A contaminated singleton read can arise from single-index hopping or double-index hopping events. The contaminated singleton reads can lead to misalignment of reads, inaccurate sequencing results, incorrect assumptions in downstream analyses, or some combination thereof. Index hopping can result from contamination from free adapters/primers that were not properly removed after adapter ligation during a cleaning step of the libraries, which are usually cleaned up by a bead-based or gel purification step to remove free adapters or primers.

Other issues may arise during the sequencing process. One such issue is pad hopping which is a form of contamination of nearby wells in a flow cell. Pad hopping leads to mixed clusters or identical sequences in nearby wells. Pad hopping duplicate reads are sequence reads that are PCR duplicates, that are co-localized. Optical duplicates can also arise from pad hopping, e.g., a strand of DNA can “hop” from an already seeded well to a nearby well, usually when insufficient DNA loading occurs, resulting in identical clusters. Clustering duplicates can also arise as an effect of pad hopping. Clustering duplicates occur when two or more nearby wells are populated with the same original fragment.

A sequencer may be configured for multiplex sequencing. In complement, the analytics system may be configured for demultiplexing. Multiplexing enables pooling and sequencing of multiple libraries simultaneously during a single sequencing run through addition of unique index sequences to each DNA fragment during library preparation. Sequencing reads are sorted to their respective samples during demultiplexing, allowing for proper alignment.

In some other examples, methods include determining whether to exclude singletons. In such cases, singletons that are present are not removed by default. For example, if WGBS is used to generate the sequencing data, then do not remove singletons by default because WGBS samples have almost all singletons.

On the other hand, if multiplex sequencing is used to generate the sequencing data, then singletons can be removed by default. This is because, presumably, double-index hopping during library multiplex enrichment is responsible for contaminated singleton reads. In such cases, double-index hopping can be used as evidence for singleton reads being contamination (e.g., contaminated singletons) that qualify for filtering out. For instance, methods can include determining to exclude singletons when they are identified as contaminated singletons, wherein a contaminated singleton is characterized by a double index hop (i.e., both indices of the singleton are swapped with the indices of another sample).

Still, in some examples, whether singletons are removed can depend on an expected error rate of the sequencing. For example, if there is a high expected error rate (e.g., expected error rate exceeds a threshold error rate), then remove singletons. The singleton read error rate increases and becomes more homogeneous at high MAF bins.

Methods for identifying double-index hopping events can include determining whether a duplicate is a sequencing duplicate or a PCR duplicate caused by PCR amplification. Double-index hopping events are recognized when either there is only one copy of a sequence read (i.e., singleton), or when there is a signature duplicate indicated by neighboring physical coordinates in the flow cell. For instance, if a duplicate read is found to originate from nearby cluster locations on the flow cell, then the duplicate happened during sequencing and is determined to be a pad-hopping duplicate read, which is filtered. On the other hand, PCR duplicates have no spatial relationship on the flow cell.

FIG. 7 illustrates an exemplary flowchart illustrating a process 700 of identifying index hopping events, according to one or more embodiments. The process 700 may be performed by the analytics system in the demultiplexing process. The process 700 applies to double-index sequencing, i.e., utilizing two indices ligated to both ends of NA fragments.

The analytics system filters 710 out single-index hopping events. The analytics system identifies mismatched index sequences in a sequence read. In a dual-indexed system, each sequencing library comprises a pair of indices. The indices in the pair are substantially unique from other indices used in other sequencing libraries. The analytics system identifies a mismatch in the index sequences when one index sequence is for a third index not part of the pair of indices in the sequencing library. For example, a first sequencing library comprises a first index for the 5′ end and a second index for the 3′ end. A sequence read that has index sequences for the first index at the 5′ end but a third index, different than the second index, at the 3′ end would be determined to be a single index-hopping event.

The analytics system collates 720 sequence reads with matched indices. To demultiplex the sequence reads, the analytics system collates sequence reads with matched indices into individual bags. For example, if a plurality of mixed sequence reads belongs to 12 different samples, each sample with a different pair of indices, then the analytics system looks to the index sequences in the sequence reads to identify the 12 different pairs of indices. Sequence reads with the same pair of indices are grouped in a bag.

The analytics system removes 730 pad-hopping duplicate reads. Pad-hopping duplicate reads are identified as being within some proximity to one another in the patterned flow cell. Each sequence read may comprise spatial information indicating a location of each sequence read on the flow cell, the spatial information comprising at least one of a lane ID, a column ID, a tile ID, or an x-y coordinate pair. To identify pad-hopping duplicate reads, the analytics system may identify a group of identical or nearly-identical sequence reads. With those identical or nearly-identical sequence reads, the analytics system determines whether the reads are co-localized.

Co-localized reads may occur when at least one of the following positional relationships is met: the grouped reads share a common tile, the grouped reads are located in neighboring tiles, the grouped reads are located in different tiles within a common column, the grouped reads are located within a threshold x-distance and a threshold y-distance from each other on the flow cell, and the grouped reads are located within a predefined boundary region. Co-localized identical or nearly-identical reads are determined to be pad-hopping duplicate reads. The predefined boundary region comprises a geometric shape having an x distance of 7,500 flow cell position units and a y distance of 100,000 flow cell position units. The geometric shape comprises a rectangle, wherein a longer side of the rectangle extends longitudinally along a lane in a y-direction of the flow cell. The threshold x-distance between the grouped reads is within a range of 0-50 mm, and the threshold y-distance between the grouped reads is within a range of 0-50 mm. The analytics system may remove the pad-hopping duplicate reads when an expected error rate associated with the multiplex sequencing exceeds a threshold error rate. The analytic system may calculate the expected error rate by: spiking the flow cell with nonhuman sequences prior to the multiplex sequencing, and determining a rate at which sequence reads corresponding to the nonhuman-based sequence mix with the sequence reads corresponding to the plurality of biological samples.

The analytics system identifies 740 double-index hopping events as singletons with unique sequences among all the other sequence reads collated based on indices. The analytic system may calculate a distance between two sequence reads based on nucleotide sequences, alignment, length of the sequence read, diversity of the sequence, etc. As an efficient approach, the analytics system may first align sequence reads together, then calculate distances between overlapping sequence reads. The analytics system determines a sequence read to be a singleton when the distance from all other sequence reads collated for the sample is above a threshold distance. As an example of de-duping, the analytics system creates new bags for each distinct sequence read. When a sequence read is greater than a threshold distance from pre-existing bags, the analytics system creates a new bag for that sequence read. Upon sorting all sequence reads, singletons are the sequence reads in their own bag, i.e., bag size of 1, without any other duplicate sequence reads. The analytics system may proceed with remedial measures, e.g., removing the sequence read having a double-index hopping event from downstream analyses.

### III. Cancer Classifier for Determining Cancer

Cancer classification involves extraction genetic features and applying one or more models to the extracted features to determine a cancer prediction. The extracted features a feature vector for a test sample and determines a cancer prediction based on the input feature vector. The cancer prediction may comprise a label and/or a value. The label may be binary, indicating a presence or absence of cancer in the test subject, and/or multiclass, indicating one or more particular cancer types from a plurality of screened cancer types. In particular, a cancer classifier may be a machine-learned model comprising a plurality of classification parameters and a function representing a relation between the feature vector as input and the cancer prediction as output. Inputting the feature vector into the function with the classification parameters yields the cancer prediction. In one or more embodiments, an age covariate prediction model is used to predict an age of the test sample based on methylation features. A residual of the predicted age and a reported age of the test subject may be utilized as a feature in the cancer classifier. In one or more embodiments, the feature vectors input into the cancer classifier are based on set of informative fragments (also referred to as “unusual fragments of extreme methylation” (UFXM)) determined from the test sample. Prior to deployment of the cancer classifier, the analytics system trains the cancer classifier. Moreover, prior to training, the analytics system may apply the sample contamination detection workflow to detect and remedy contamination events from the multiplex sequencing data.

### III.A. Identifying Informative Fragments

The analytics system can determine informative fragments for a sample using the sample's methylation state vectors. For each fragment in a sample, the analytics system can determine whether the fragment is an informative fragment using the methylation state vector corresponding to the fragment. In some embodiments, the analytics system calculates a p-value score for each methylation state vector describing a probability of observing that methylation state vector or other methylation state vectors even less probable in the healthy control group. The process for calculating a p-value score is further discussed below in Section III.C.i. P-Value Filtering. The analytics system may determine fragments with a methylation state vector having below a threshold p-value score as informative fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining informative fragments. Examples of other probabilistic models include a mixture model, a deep probabilistic model, etc. In some embodiments, the analytics system may use any combination of the processes described below for identifying informative fragments. With the identified informative fragments, the analytics system may filter the set of methylation state vectors for a sample for use in other processes, e.g., for use in training and deploying a cancer classifier.

### III.A.I. P-Value Filtering

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be informatively methylated, the analytics system can use a healthy control group with a majority of fragments that are normally methylated. When conducting this probabilistic analysis for determining informative fragments, the determination can hold weight in comparison with the group of control subjects that make up the healthy control group. To ensure robustness in the healthy control group, the analytics system may select some threshold number of healthy individuals to source samples including DNA fragments. FIG. 8A below describes the method of generating a data structure for a healthy control group with which the analytics system may calculate p-value scores. FIG. 8B describes the method of calculating a p-value score with the generated data structure.

FIG. 8A is a flowchart describing a process 800 of generating a data structure for a healthy control group, according to an embodiment. To create a healthy control group data structure, the analytics system can receive a plurality of DNA fragments (e.g., cfDNA) from a plurality of healthy individuals. A methylation state vector can be identified for each fragment, for example via the process 300.

With each fragment's methylation state vector, the analytics system can subdivide 205 the methylation state vector into strings of CpG sites. In some embodiments, the analytics system subdivides 205 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to x4 can result in x4 strings of length x4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 810 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 810 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 815 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of x4 means that every CpG site has at the very least 2\hat{ }4 numbers to tally for strings of length x4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of informativeness that predictive of the presence of cancer), calculating probabilities based on large strings of CpG sites can be problematic as it uses a significant amount of data that may not be available, and thus can be too sparse for a model to perform appropriately. For example, calculating a probability of informativeness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is informative or not.

FIG. 8B is a flowchart describing a process 820 for identifying informatively methylated fragments from an individual, according to an embodiment. In process 820, the analytics system generates 200 methylation state vectors from cfDNA fragments of the subject. The analytics system can handle each methylation state vector as follows.

For a given methylation state vector, the analytics system enumerates 830 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2″ possibilities of methylation state vectors. With methylation state vectors inclusive of indeterminate states for one or more CpG sites, the analytics system may enumerate 830 possibilities of methylation state vectors considering only CpG sites that have observed states.

The analytics system calculates 840 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMMs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 850 a p-value score for the methylation state vector using the calculated probabilities for each possibility. In some embodiments, this includes identifying the calculated probability corresponding to the possibility that matches the methylation state vector in question. Specifically, this can be the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy individual, and which causes the fragment to be labeled informatively methylated, relative to the healthy control group. A high p-value score can generally relate to a methylation state vector is expected to be present, in a relative sense, in a healthy individual. If the healthy control group is a non-cancerous group, for example, a low p-value can indicate that the fragment is informative methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are informatively methylated, the analytics system may filter 860 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 800, the analytics system can yield a median (range) of 2,800 (1,500-12,000) fragments with informative methylation patterns for participants without cancer in training, and a median (range) of 3,000 (1,200-420,000) fragments with informative methylation patterns for participants with cancer in training. These filtered sets of fragments with informative methylation patterns may be used for the downstream analyses as described below in Section III.

In some embodiments, the analytics system uses 855 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytic system can calculate a p-value score for the window including the first CpG site. The analytics system can then “slide” the window to the second CpG site in the vector, and calculates another p-value score for the second window.

Thus, for a window size/and methylation vector length m, each methylation state vector can generate m−l+l p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of informative fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of 1's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. Provisional patent application Ser. No. 17/191,914, titled “Systems and Methods for Cancer Condition Determination Using Autoencoders,” filed Mar. 4, 2021, which is hereby incorporated herein by reference in its entirety.

The respective nucleic acid methylation fragment may fail to satisfy a selection criterion in the one or more selection criteria when the respective nucleic acid methylation fragment has an informative methylation score that is less than an informative methylation score threshold. In this situation, the informative methylation score can be determined by a mixture model. For example, a mixture model can detect an informative methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An informative methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 8, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 8, 5, or more than 5. For example, a first nucleic acid methylation fragment having the same corresponding genomic start and end position as a second nucleic acid methylation fragment but having at least 1, at least 2, at least 3, at least 8, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

### III.A.II. Hypermethylated Fragments and Hypomethylated Fragments

In some embodiments, the analytics system determines informative fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

### III.B. Training of Cancer Classifier

FIG. 9A is a flowchart describing a process 900 of training a cancer classifier, according to an embodiment. The analytics system obtains 910 a plurality of training samples each having a set of informative fragments and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 920, for each training sample, a feature vector based on the set of informative fragments of the training sample. The analytics system can calculate an informative score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the informative score for the feature vector with a binary scoring based on whether there is an informative fragment in the set of informative fragments that encompasses the CpG site. In another embodiment, the analytics system defines the informative score based on a count of informative fragments overlapping the CpG site. In one example, the analytics system may use a trinary scoring assigning a first score for lack of presence of informative fragments, a second score for presence of a few informative fragments, and a third score for presence of more than a few informative fragments. For example, the analytics system counts x5 informative fragment in a sample that overlap the CpG site and calculates an informative score based on the count of x5.

Once all informative scores are determined for a training sample, the analytics system can determine the feature vector as a vector of elements including, for each element, one of the informative scores associated with one of the CpG sites in an initial set. The analytics system can normalize the informative scores of the feature vector based on a coverage of the sample. Here, coverage can refer to a median or average sequencing depth over all CpG sites covered by the initial set of CpG sites used in the classifier, or based on the set of informative fragments for a given training sample.

As an example, reference is now made to FIG. 9B illustrating a matrix of training feature vectors 922. In this example, the analytics system has identified CpG sites [K] 926 for consideration in generating feature vectors for the cancer classifier. The analytics system selects training samples [N] 924. The analytics system determines a first informative score 928 for a first arbitrary CpG site [k1] to be used in the feature vector for a training sample [n1]. The analytics system checks each informative fragment in the set of informative fragments. If the analytics system identifies at least one informative fragment that includes the first CpG site, then the analytics system determines the first informative score 928 for the first CpG site as 1, as illustrated in FIG. 9B. Considering a second arbitrary CpG site [k2], the analytics system similarly checks the set of informative fragments for at least one that includes the second CpG site [k2]. If the analytics system does not find any such informative fragment that includes the second CpG site, the analytics system determines a second informative score 929 for the second CpG site [k2] to be 0, as illustrated in FIG. 9B. Once the analytics system determines all the informative scores for the initial set of CpG sites, the analytics system determines the feature vector for the first training sample [n1] including the informative scores with the feature vector including the first informative score 928 of 1 for the first CpG site [k1] and the second informative score 929 of 0 for the second CpG site [k2] and subsequent informative scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety.

The analytics system may further limit the CpG sites considered for use in the cancer classifier. The analytics system computes 930, for each CpG site in the initial set of CpG sites, an information gain based on the feature vectors of the training samples. From step 920, each training sample has a feature vector that may contain an informative score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 930 an information gain for each cancer type and for each CpG site in the initial set to determine whether to include that CpG site in the classifier. The information gain is computed for training samples with a given cancer type compared to all other samples. For example, two random variables ‘informative fragment’ (‘IF’) and ‘cancer type’ (‘CT’) are used. In one embodiment, IF is a binary variable indicating whether there is an informative fragment overlapping a given CpG site in a given samples as determined for the informative score/feature vector above. CT is a random variable indicating whether the cancer is of a particular type. The analytics system computes the mutual information with respect to CT given AF. That is, how many bits of information about the cancer type are gained if it is known whether there is an informative fragment overlapping a particular CpG site. In practice, for a first cancer type, the analytics system computes pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly informatively methylated in training samples of a given cancer but not in training samples of other cancer types or in healthy training samples, then CpG sites overlapped by those informative fragments can have high information gains for the given cancer type. The ranked CpG sites for each cancer type can be greedily added (selected) 940 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 950 the feature vectors of the training samples as needed. For example, the analytics system may truncate feature vectors to remove informative scores corresponding to CpG sites not in the selected set of CpG sites.

With the feature vectors of the training samples, the analytics system may train the cancer classifier in any of a number of ways. The feature vectors may correspond to the initial set of CpG sites from step 920 or to the selected set of CpG sites from step 950. In one embodiment, the analytics system trains 960 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 970 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

### III.C. Deployment of Cancer Classifier

During use of the cancer classifier, the analytics system can obtain a test sample from a subject of unknown cancer type. The analytics system may process the test sample comprised of DNA molecules with any combination of the processes 300, 400, and 420 to achieve a set of informative fragments. The analytics system can determine a test feature vector for use by the cancer classifier according to similar principles discussed in the process 500. The analytics system can calculate an informative score for each CpG site in a plurality of CpG sites in use by the cancer classifier. For example, the cancer classifier receives as input feature vectors inclusive of informative scores for 1,000 selected CpG sites. The analytics system can thus determine a test feature vector inclusive of informative scores for the 1,000 selected CpG sites based on the set of informative fragments. The analytics system can calculate the informative scores in a same manner as the training samples. In some embodiments, the analytics system defines the informative score as a binary score based on whether there is a hypermethylated or hypomethylated fragment in the set of informative fragments that encompasses the CpG site.

The analytics system can then input the test feature vector into the cancer classifier. The function of the cancer classifier can then generate a cancer prediction based on the classification parameters trained in the process 500 and the test feature vector. In the first manner, the cancer prediction can be binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 960 of the process 900 with another cancer classifier trained in step 970 or the process 900. The analytics system can input the test feature vector into the cancer classifier trained as a binary classifier in step 960 of the process 900. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

### IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

### IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

### IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V.D. Kit Implementation

Also disclosed herein are kits for performing the methods described above including the methods relating to the cancer classifier. The kits may include one or more collection vessels for collecting a sample from the individual comprising genetic material. The sample can include blood, plasma, serum, urine, fecal, saliva, other types of bodily fluids, or any combination thereof. Such kits can include reagents for isolating nucleic acids from the sample. The reagents can further include reagents for sequencing the nucleic acids including buffers and detection agents. In one or more embodiments, the kits may include one or more sequencing panels comprising probes for targeting particular genomic regions, particular mutations, particular genetic variants, or some combination thereof. In other embodiments, samples collected via the kit are provided to a sequencing laboratory that may use the sequencing panels to sequence the nucleic acids in the sample. The WBC contamination detection may be applied to various configurations of the kit, to minimize WBC contamination potentially originating from components of the kit. For example, experiments may be run comparing types of collection vessels. WBC contamination can be assessed and compared between the types of collection vessels to identify an optimal type that minimizes WBC contamination.

A kit can further include instructions for use of the reagents included in the kit. For example, a kit can include instructions for collecting the sample, extracting the nucleic acid from the test sample. Example instructions can be the order in which reagents are to be added, centrifugal speeds to be used to isolate nucleic acids from the test sample, how to amplify nucleic acids, how to sequence nucleic acids, or any combination thereof. The instructions may further illumine how to operate a computing device as the analytics system 200, for the purposes of performing the steps of any of the methods described.

In addition to the above components, the kit may include computer-readable storage media storing computer software for performing the various methods described throughout the disclosure. One form in which these instructions can be present is as printed information on a suitable medium or substrate, e.g., a piece or pieces of paper on which the information is printed, in the packaging of the kit, in a package insert. Yet another means would be a computer readable medium, e.g., diskette, CD, hard-drive, network data storage, on which the instructions have been stored in the form of computer code. Yet another means that can be present is a website address or QR code which can be used via the internet to access the information at a removed site.

### V.F. Contamination Source Detection and Mitigation

In some embodiments, the methods and/or classifier of the present invention are used to detect sample contamination, e.g., as described by the various methodologies in Sections II.C. & II.D.

The analytics system may leverage the WBC contamination workflow to identify a source of the contamination. To identify the source, the analytics system may isolate one or more variables of the sample processing workflow. The analytics system may process a first of samples with a first sample processing workflow and a second set of samples with a second sample processing workflow, with the second sample processing workflow different in the one or more variables being assessed. For example, the second sample processing workflow may include a different protocol, a different clinical product, or a different sequencing device. Protocols may include steps undertaken in processing the sample, e.g., centrifugation, storage temperature, storage duration, etc. Clinical products are manufactured products used in the sample processing workflow and may include, e.g., any vessel, any chemical, any compound, any buffer, any solution, any enzyme, or any other product used in the workflow. The sequencing device may generally include the sequencer, but may also include other devices related to the sequencing process. The sample processing workflow may further include other laboratory devices, e.g., centrifuge, storage devices, other laboratory devices for sample processing, etc.

The analytics system applies a contamination model to the samples from both sample processing workflows. The analytics system may determine an aggregate metric for each sample processing workflow based on the contamination results of the respective sample sets. If there is a significant difference in aggregate metrics between the sample processing workflows, then the analytics system may identify the source based on what variables were different between the first and the second sample processing workflows. Remedial measures may also be implemented.

With the contamination source identified, the analytics system may determine an optimal sample processing workflow that mitigates the WBC contamination. For example, through iterative testing, the analytics system may determine a set of protocols that minimizes contamination, a set of clinical products that minimizes the WBC contamination, a set of one or more sequencing devices that minimizes the WBC contamination, or some combination thereof. The optimal sample processing workflow may then be applied to subsequent samples.

### V. Example Results

### V.A. Sample Collection and Processing

Study design and samples: CCGA (NCT02889978) is a prospective, multi-center, case-control, observational study with longitudinal follow-up. De-identified biospecimens were collected from approximately 15,000 participants from 342 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, MD). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, MI) and constructed libraries were quantified using KAPA Library Quantification Kit for Illumina Platforms (Kapa Biosystems; Wilmington, MA). Four libraries along with 10% PhiX v3 library (Illumina, FC-110-3001) were pooled and clustered on an Illumina NovaSeq 7000 S2 flow cell followed by 150-bp paired-end sequencing (30×).

For each sample, the WGBS fragment set was reduced to a small subset of fragments having an informative methylation pattern. Additionally, hyper or hypomethylated cfDNA fragments were selected. cfDNA fragments selected for having an informative methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.C. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypo-methylated). This procedure resulted in a median (range) of 2,800 (1,500-12,000) UFXM fragments for participants without cancer in training, and a median (range) of 3,000 (1,200-420,000) UFXM fragments for participants with cancer in training. As this data reduction procedure only used reference set data, this stage was only required to be applied to each sample once.

### V.B. Sample Contamination Detection Results

According to one example experiment, the contamination detection process was able to determine that 88% of singleton reads were dual-index hopping events. The experiment was conducted on 36 individual cfDNA samples utilizing 36 unique sample barcodes. Dual-index hopped fragments are expected to be all singletons, but not all singletons are generated by double index hopping. The below table illustrates the number of sequence reads affirmatively identified as due to double index hopping.

TABLE 1
Sample Barcode Detection of Known Contamination Reads

| Detected by Sample Barcode? | Bag Size | Count |
|---|---|---|
| Yes | Singleton | 79 |
| No | Singleton | 11 |
| Yes | Non-singleton | 0 |
| No | Non-singleton | 56 |

In total, there were 90 singleton sequence reads and 56 non-singleton sequence reads known to be contaminated. The contamination could be due to double index hopping, sequencing errors, sample contamination during collection, processing, etc. In the case of the singleton sequence reads, the contamination detection process using sample barcodes was able to affirmatively identify 79 out of the 90 contaminated reads as dual-index hopping events. With the sample barcode, the analytics system could return those reads to the appropriate sample.

### VI. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method comprising:
ligating one of a plurality of molecule identifiers (MIs) to a first end of each nucleic acid (NA) fragment of a first sample, wherein at least two of the plurality of MIs are different from one another;
amplifying the NA fragments to produce amplified NA fragments comprising one or more copies of each NA fragment;
ligating a first sample barcode to the amplified NA fragments of the first sample;
indexing the amplified NA fragments to produce indexed NA fragments each comprising a first index and a second index;
sequencing the indexed NA fragments to generate a sequence read for each indexed NA fragment;
collecting sequence reads of indexed NA fragments each comprising a first index sequence read and a second index sequence read in a group; and
identifying a contamination event for a first sequence read in the group by identifying a second sample barcode that is different than the first sample barcode on the sequence read.

2. The method of claim 1, wherein each amplified NA fragment having the first sample barcode comprises a target NA region derived from the sample and wherein each amplified NA fragment having the second sample barcode comprises a target NA region derived from a second sample that is different than the first sample.

3. The method of claim 1, further comprising removing the first sequence read with the index hopping event from the group for the first sample.

4. (canceled)

5. (canceled)

6. The method of claim 1, wherein the one of the plurality of MIs and the NA fragment are single-stranded during ligation.

7.-9. (canceled)

10. The method of claim 1, wherein the first sample barcode is ligated to a second end of the amplified NA fragments opposite the first end.

11. The method of claim 1, wherein the first sample barcode is ligated to the first end of the amplified NA fragments, adjacent to the one of the plurality of MIs.

12. The method of claim 1, wherein the first index is ligated to the first end, and the second index is ligated to a second end that is opposite the first end.

13. The method of claim 1, wherein the first index and the second index are ligated to the first end of the amplified NA fragments.

14. The method of claim 1, wherein the first index and the second index are ligated to a second end of the amplified NA fragments that is opposite the first end.

15. The method of claim 1, wherein the sequencing is multiplexed with a plurality of samples across a plurality of flow cells, and wherein the first index and the second index are used for a first column that the first sample is in.

16.-35. (canceled)

36. A method for processing sequencing data, comprising:
receiving sequencing data comprising a set of sequence reads generated from multiplex sequencing a plurality of biological samples, each containing nucleic acid, the sequencing data including data generated from single-index hopping and double-index hopping events arising during the multiplex sequencing;
filtering the sequencing data to exclude data corresponding to the single-index hopping events, the filtering comprising:
identifying, in the set of sequence reads, one or more reads having a mismatched pair of indices, the mismatched pair of indices comprising two unique indices corresponding to two different biological samples; and
filtering the sequencing data to exclude data corresponding to the double-index hopping events, the filtering comprising:
identifying, in the set of sequence reads, one or more pad-hopping duplicate reads, the pad-hopping duplicate reads having duplicate sequences that are co-localized in a flow cell used during the multiplex sequencing; and
subsequent to identifying the one or more pad-hopping duplicate reads, identifying, in the set of sequence reads, one or more singletons, each singleton comprising a unique sequence read among the set of sequence reads.

37. The method of claim 36, further comprising:
removing the identified one or more pad-hopping duplicate reads from the set of sequencing reads; and
subsequent to removing the identified one or more pad-hopping duplicate reads, identifying the one or more singletons in the remaining set of sequencing reads.

38. (canceled)

39. (canceled)

40. The method of claim 36, wherein the flow cell comprises a plurality of physically separated lanes, wherein each lane comprises multiple columns with each column comprising a plurality of tiles, further wherein each lane defines a surface having a plurality of wells arranged thereon.

41. (canceled)

42. The method of claim 36, wherein identifying pad-hopping duplicate reads comprises:
identifying a group of identical or nearly-identical sequence reads;
determining, based on the sequence data, whether the grouped reads are co-localized, wherein the grouped reads are co-localized when at least one of the following positional relationships is met:
the grouped reads share a common tile,
the grouped reads are located in neighboring tiles,
the grouped reads are located in different tiles within a common column,
the grouped reads are located within a threshold x-distance and a threshold y-distance from each other on the flow cell, and
the grouped reads are located within a predefined boundary region; and
in accordance with a determination that the grouped reads are co-localized, identifying the grouped reads as pad-hopping duplicate reads.

43.-46. (canceled)

47. The method of claim 36, comprising:
removing the pad-hopping duplicate reads when an expected error rate associated with the multiplex sequencing exceeds a threshold error rate.

48. The method of claim 36, comprising:
providing the filtered sequencing data for analysis using a statistical model, wherein a limit of detection associated with the filtered sequencing data is lower than a limit of detection associated with unfiltered sequencing data.

49. (canceled)

50. (canceled)

51. The method of claim 36, further comprising:
fragmenting the nucleic acid extracted from the plurality of biological samples into genomic fragments;
ligating unique dual index pairs to end portions of the genomic fragments to generate multiple library fragments, wherein each unique dual index pair identifies an individual biological sample in the plurality of biological samples;
enriching the library fragments by capturing certain library fragments with targeted probes and amplifying the captured library fragments within multiple wells on the flow cell, wherein each well is configured to hold a clonal cluster of amplified fragments originating from a single library fragment;
sequencing the enriched fragments to produce the sequencing data comprising the set of sequence reads, each sequence read comprising a plurality of nucleotide base calls; and
demultiplexing the set of sequence reads based on the unique dual index pairs to determine the original biological sample for each sequence read.

52. The method of claim 51, further comprising demultiplexing the sequencing data after filtering the sequencing data to exclude the data corresponding to the single-index hopping events and double-index hopping events.

53. (canceled)

54. A method for training a cancer classifier comprising:
performing next-generation multiplex sequencing of a set of training samples each with a known cancer state to obtain a plurality of sequence reads of amplified fragments, wherein each sequence read comprises a pair of indices and a sample barcode ligated onto a target region of a nucleic acid fragment;
collating the sequence reads into a plurality of bags based on the pairs of indices of the sequence reads, wherein each bag includes sequence reads having a common pair of indices;
detecting a cross-sample contamination event for a first sequence read having a first sample barcode different from other sequence reads in a first bag;
removing the first sequence read from the first bag;
assigning remaining sequence reads in each bag to one training sample;
determining a feature vector for each training sample based on the sequence reads in the corresponding bag; and
training the cancer classifier with the feature vectors for the training samples, wherein the trained cancer classifier is configured to predict likelihood of presence of cancer based on an input feature vector derived based on sequence reads in a test sample.

55. The method of claim 54, wherein each sequence read further comprises a molecular identifier (MI) ligated onto the corresponding nucleic acid fragment, and wherein the method further comprises:
collapsing sequences reads in each bag into distinct sequence reads based on the molecular identifiers, wherein sequence reads overlapping a similar genomic position and having matching molecular identifiers are determined to read on the same nucleic acid fragment.

56. The method of claim 54, further comprising:
assigning the first sequence read to a second bag with sequence reads in the second bag having the first sample barcode.

57. (canceled)

58. The method of claim 54, wherein the cancer classifier is a machine-learning model trained to predict a binary prediction between presence of cancer or absence of cancer, a multiclass prediction as a likelihood of presence of one of a plurality of cancer types, or some combination thereof.

59.-76. (canceled)
